(19) United States
(12) Patent Application Publication
(10) Pub. No.: US 2016/0118595 A1
(43) Pub. Date: Apr. 28, 2016

(54) MATERIAL FOR USE IN ORGANIC ELECTROLUMINESCENT DEVICE AND ORGANIC ELECTROLUMINESCENT DEVICE USING THE SAME

(71) Applicant: SAMSUNG DISPLAY CO., LTD., Yongin-si (KR)

(72) Inventor: Hiroaki Itoi, Yokohama (JP)

(21) Appl. No.: 14/864,612

(22) Filed: Sep. 24, 2015

(30) Foreign Application Priority Data
Oct. 22, 2014 (JP) 2014-215662

(51) Int. Cl. H01L 51/00; C07D 471/04

(52) U.S. Cl. CPC H01L 51/0061; C07D 471/04; H01L 51/0072; H01L 51/5206

(57) ABSTRACT

An organic electroluminescent device includes an anode, a cathode, an emission layer between the anode and the cathode, and a plurality of lamination layers between the emission layer and the anode, wherein at least one of the plurality of lamination layers includes a material for an organic electroluminescent device represented by the following Formula 1. The organic electroluminescent device using the material may have improved lifetime.

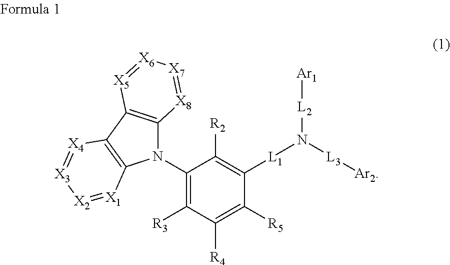

### CROSS-REFERENCE TO RELATED APPLICATION

This application claims priority to and the benefit of Japanese Patent Application No. 2014-215662, filed on Oct. 22, 2014, the entire content of which is hereby incorporated by reference.

### BACKGROUND

One or more aspects of embodiments of the present disclosure relate to a material for an organic electroluminescent device and an organic electroluminescent device using the same, and more particularly, to a hole transport material for an organic electroluminescent device which has a long lifetime, and an organic electroluminescent device using the same.

Recently, organic electroluminescent (EL) displays have been actively developed as display devices. Organic EL displays differ from displays such as, for example, liquid crystal displays, and are self-emitting displays in which light is generated by light-emitting materials, including organic compounds, upon recombination of holes and electrons respectively injected from an anode and a cathode into an emission layer.

An example of an organic electroluminescent device (hereinafter, organic EL device), may include an anode, a hole transport layer on the anode, an emission layer on the hole transport layer, an electron transport layer on the emission layer, and a cathode on the electron transport layer. Holes are injected from the anode, travel through the hole transport layer, and are injected into the emission layer. Electrons are injected from the cathode, travel through the electron transport layer, and are injected into the emission layer. The holes and electrons that are injected into the emission layer are recombined to generate excitons. Organic electroluminescent devices emit light through the radiative decay of these excitons. Organic electroluminescent devices are not limited to the above-described configuration, and many modifications thereof are possible.

The organic electroluminescent devices that are utilized in displays may require improved efficiencies and longer lifetimes. For example, organic electroluminescent devices in the blue emission region may exhibit comparatively higher operating voltages and lowered light emission efficiencies when compared to those in the green and red emission regions. Strategies such as normalization, stabilization, and improved durability of the hole transport layer are being investigated in order to realize organic electroluminescent devices having improved efficiencies and longer lifetimes.

Although various hole transport materials such as, for example, aromatic amine-based compounds have been used in the hole transport layer, they may limit the device lifetime. For example, in Japanese Patent Application Laid-open Publication No. 2009-267255 A and Japanese Patent Application Laid-open Publication No. 2009-029726 A, aryl and heteroaryl group-substituted amine derivatives are proposed as materials that are advantageous for improving the lifetimes of organic electroluminescent devices. However, even the organic electroluminescent devices which use the above-mentioned materials cannot be considered to have an emission lifetime that is sufficiently long enough for use in an ideal display. Therefore, an organic electroluminescent device with an even longer emission lifetime is presently desirable.

### SUMMARY

One or more aspects of embodiments of the present disclosure are directed toward a material for an organic electroluminescent device which has a long lifetime, and an organic electroluminescent device using the same.

In some embodiments, the present disclosure provides a material for use in an organic electroluminescent device which emits light in the green to blue emission regions, has a long lifetime, and which is included in at least one lamination layer positioned between an emission layer and an anode of an organic electroluminescent device.

One or more embodiments of the present disclosure provide a material for an organic electroluminescent device, represented by the following Formula 1:

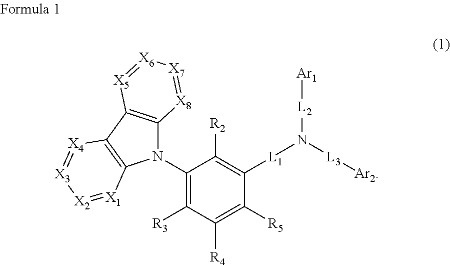

In the above Formula 1, X₁ to X₈ may each independently be selected from N and CR₁, and at least one of X₁ to X₈ may be N; R₁ to R₅ may be each independently selected from a substituted or unsubstituted aryl group having 6 to 30 carbon atoms for forming a ring, a substituted or unsubstituted heteroaryl group having 1 to 30 carbon atoms for forming a ring, an alkyl group having 1 to 15 carbon atoms, a substituted or unsubstituted silyl group, a halogen atom, hydrogen, and deuterium; L₁ to L₃ may be each independently a single bond or a divalent group selected from a substituted or unsubstituted alkylene group, a substituted or unsubstituted aralkylene group, a substituted or unsubstituted arylene group, a substituted or unsubstituted heteroarylene group, and a silyl group; and Ar₁ and Ar₂ may be each independently selected from a substituted or unsubstituted aryl group having 6 to 30 carbon atoms for forming a ring and a substituted or unsubstituted heteroaryl group having 1 to 30 carbon atoms for forming a ring.

One or more embodiments of the present disclosure provide a material for an organic electroluminescent device, in which an azacarbazole part (e.g., azacarbazole moiety) is coupled to a meta (m) position of a phenylene group which is bonded, directly (e.g. via a bond such as a single bond) or through L₁, to an amine moiety. Consequently, the degree of amorphousness of the material is increased along with the carrier mobility (e.g., hole mobility). Furthermore, since the azacarbazole part (e.g., azacarbazole moiety) contributes to improving the electron resistance of the material, a long lifetime of the organic electroluminescent device may be realized. For example, improved characteristics may be obtained in the green to blue emission regions.

One or more embodiments of the present disclosure provide a material for an organic electroluminescent device, represented by the following Formula 2:

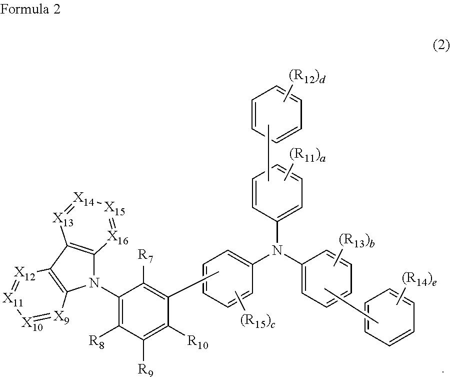

In the above Formula 2, X₉ to X₁₆ may be each independently selected from N and CR₆, and at least one of X₉ to X₁₆ may be N; R₆ to R₁₅ may be each independently selected from a substituted or unsubstituted aryl group having 6 to 30 carbon atoms for forming a ring, a substituted or unsubstituted heteroaryl group having 1 to 30 carbon atoms for forming a ring, an alkyl group having 1 to 15 carbon atoms, a substituted or unsubstituted silyl group, a halogen atom, hydrogen, and deuterium; a to c may be each independently an integer selected from 0 to 4, and d and e may be each independently an integer selected from 0 to 5.

One or more embodiments of the present disclosure provide a material for an organic electroluminescent device as represented by Formula 1, in which Ar₁ and Ar₂ may each independently include a substituted or unsubstituted phenyl group, and L₁ to L₃ may each independently include a substituted or unsubstituted phenylene group. Conjugation of the resulting amine may enhance the charge resistance of the material.

One or more embodiments of the present disclosure provide an organic electroluminescent device that includes the material for an organic electroluminescent device in at least one lamination layer positioned between an emission layer and an anode.

One or more embodiments of the present disclosure provide an organic electroluminescent device capable of realizing a long lifetime through the use of the material for an organic electroluminescent device, which is included in at least one lamination layer positioned between an emission layer and an anode. For example, improved characteristics may be obtained in the green to blue emission regions.

### BRIEF DESCRIPTION OF THE FIGURES

The accompanying drawings are included to provide a further understanding of the present disclosure, and are incorporated in and constitute a part of this specification. The drawings illustrate example embodiments and explain principles of the present disclosure In the drawings:

FIG. 1 is a schematic diagram illustrating an organic electroluminescent device according to one or more embodiments of the present disclosure; and

FIG. 2 is a schematic diagram illustrating an organic electroluminescent device according to one or more embodiments of the present disclosure.

### DETAILED DESCRIPTION

According to one or more embodiments of the present disclosure, a material for an organic electroluminescent device may realize long lifetime and high emission by introducing an azacarbazole part (e.g., azacarbazole moiety) coupled to a meta position of a phenylene group that is bonded, directly (e.g., via a bond such as a single bond) or through a connecting group, to the nitrogen atom (N) of an amine moiety, thereby improving the degree of amorphousness of the material. This results in an increase in the carrier mobility (e.g., hole mobility) and also improves the heat resistance and electron resistance,

Hereinafter, a material for an organic electroluminescent device and an organic electroluminescent device using the same, according to one of more embodiments of the present disclosure, will be described with reference to the accompanying drawings. The material for an organic electroluminescent device according to embodiments of the present disclosure and an organic electroluminescent device using the same may be embodied in many different forms and should not be construed as being limited to the embodiments set forth herein. In the drawings, like reference numerals refer to like elements or elements having like functions throughout, and repeated explanation thereof will not be provided herein.

A material for an organic electroluminescent device according to one or more embodiments of the present disclosure may include an amine compound in which an azacarbazole part (e.g., azacarbazole moiety) is introduced (e.g., coupled) to a meta position of a phenylene group which is bonded to an amine moiety, and the material may be represented by the following Formula 1:

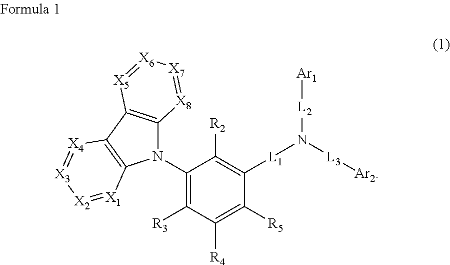

According to one or more embodiments of the present disclosure, a material for an organic electroluminescent device may be represented by Formula 1, wherein X₁ to X₈ may be each independently selected from N and CR₁, and at least one of X₁ to X₈ may be N. In one embodiment, only one of X₁ to X₈ is N, but X₁ to X₈ are not limited thereto. R₁ to R₅ may be each independently selected from a substituted or unsubstituted aryl group having 6 to 30 carbon atoms for forming a ring, a substituted or unsubstituted heteroaryl group having 1 to 30 carbon atoms for forming a ring, an alkyl group having 1 to 15 carbon atoms, a substituted or unsubstituted silyl group, a halogen atom, hydrogen, and deuterium. As used herein, the statement “atoms for forming a ring” may refer to “ring-forming atoms.” L₁ to L₃ may be each independently a single bond or a divalent group selected from, for example, a substituted or unsubstituted alkylene group, a substituted or unsubstituted aralkylene group, a substituted or unsubstituted arylene group, a substituted or unsubstituted heteroarylene group, and a silyl group; and Ar₁ and Ar₂ may be each independently selected from a substituted or unsubstituted aryl group having 6 to 30 carbon atoms for forming a ring and a substituted or unsubstituted heteroaryl group having 1 to 30 carbon atoms for forming a ring.

In Formula 1, non-limiting examples of the substituted or unsubstituted aryl group having 6 to 30 carbon atoms for forming a ring used in, for example, R₁ to R₅ may include a phenyl group, a naphthyl group, an anthracenyl group, a phenanthryl group, a biphenyl group, a terphenyl group, a quaterphenyl group, a quinqphenyl group, a sexiphenyl group, a fluorenyl group, a triphenylenyl group, a biphenylenyl group, a pyrenyl group, a benzofluoranthenyl group, a glyceryl group, and the like.

Non-limiting examples of the substituted or unsubstituted heteroaryl group having 1 to 30 carbon atoms for forming a ring used in, for example, R₁ to R₅ may include a pyridyl group, a furyl group, a thienyl group, a quinolyl group, an isoquinolyl group, a benzofuryl group, a benzothienyl group, an indolyl group, a benzoxazolyl group, a benzothiazolyl group, a quinoxalyl group, a benzoimidazolyl group, a dibenzofuryl group, a dibenzothienyl group, a carbazolyl group, and the like.

Non-limiting examples of the alkyl group having 1 to 15 carbon atoms used in, for example, R₁ to R₅ may include a methyl group, an ethyl group, a propyl group, an isopropyl group, a n-butyl group, a s-butyl group, an isobutyl group, a t-butyl group, a n-pentyl group, a n-hexyl group, a n-heptyl group, a n-octyl group, a hydroxymethyl group, a 1-hydroxyethyl group, a 2-hydroxyethyl group, a 2-hydroxyisobutyl group, a 1,2-dihydroxyethyl group, a 1,3-dihydroxyisopropyl group, a 2,3-dihydroxy-t-butyl group, a 1,2,3-trihydroxypropyl group, a chloromethyl group, a 1-chloroethyl group, a 2-chloroethyl group, a 2-chloroisobutyl group, a 1,2-dichloroethyl group, a 1,3-dichloroisopropyl group, a 2,3-dichloro-t-butyl group, a 1,2,3-trichloropropyl group, a bromomethyl group, a 1-bromoethyl group, a 2-bromoethyl group, a 2-bromoisobutyl group, a 1,2-dibromoethyl group, a 1,3-dibromoisopropyl group, a 2,3-dibromo-t-butyl group, a 1,2,3-tribromopropyl group, an iodomethyl group, a 1-iodoethyl group, a 2-iodoethyl group, a 2-iodoisobutyl group, a 1,2-diiodoethyl group, a 1,3-diiodoisopropyl group, a 2,3-diiodo-t-butyl group, a 1,2,3-triiodopropyl group, a cyanomethyl group, a 1-cyanoethyl group, a 2-cyanoethyl group, a 2-cyanoisobutyl group, a 1,2-dicyanoethyl group, a 1,3-dicyanoisopropyl group, a 2,3-dicyano-t-butyl group, a 1,2,3-tricyanopropyl group, a nitromethyl group, a 1-nitroethyl group, a 2-nitroethyl group, a 2-nitroisobutyl group, a 1,2-dinitroethyl group, a 1,3-dinitroisopropyl group, a 2,3-dinitro-t-butyl group, a 1,2,3-trinitropropyl group, a cyclopropyl group, a cyclobutyl group, a cyclopentyl group, a cyclohexyl group, a 4-methylcyclohexyl group, a 1-adamantyl group, a 2-adamantyl group, a 1-norbornyl group, a 2-norbornyl group, and the like.

In addition, non-limiting examples of the silyl group used in, for example, R₁ to R₅ may include a trialkylsilyl group, a triarylsilyl group, a monoalkyldiarylsilyl group, and a dialkylmonoarylsilyl group, and in some embodiments may include, for example, a trimethylsilyl group, a triphenylsilyl group, and the like.

In addition, the halogen atom used in, for example, R₁ to R₅ may include a fluorine atom (F), a chlorine atom (Cl), a bromine atom (Br), and the like.

In some embodiments, R₁ to R₅ may each independently be selected from hydrogen and deuterium.

In Formula 1, respective atoms in a plurality of adjacent R₁(s) and/or respective atoms in a plurality of adjacent R₃ to R₅ may be bonded (e.g., coupled) to each other to form a saturated or unsaturated ring.

In Formula 1, non-limiting examples of the substituted or unsubstituted aryl group having 6 to 30 carbon atoms for forming a ring used in, for example, Ar₁ and Ar₂ may include a phenyl group, a naphthyl group, an anthracenyl group, a phenanthryl group, a biphenyl group, a terphenyl group, a quaterphenyl group, a fluorenyl group, a triphenylenyl group, a biphenylenyl group, a pyrenyl group, a benzofluoranthenyl group, a glyceryl group, a phenylnaphthyl group, a naphthylphenyl group, and the like.

In addition, non-limiting examples of the substituted or unsubstituted heteroaryl group having 1 to 30 carbon atoms for forming a ring used in, for example, Ar₁ and Ar₂ may include a pyridyl group, a furyl group, a thienyl group, a quinolyl group, an isoquinolyl group, a benzofuryl group, a benzothienyl group, an indolyl group, a benzoxazolyl group, a benzothiazolyl group, a quinoxalyl group, a benzoimidazolyl group, a dibenzofuryl group, a dibenzothienyl group, a carbazolyl group, and the like.

In Formula 1, non-limiting examples of the substituted or unsubstituted alkylene group used in, for example, L₁ to L₃ as the divalent connecting group may include a methylene group, an ethylene group, an n-propylene group, an n-butylene group, an n-hexylene group, an n-heptylene group, an n-octylene group, an n-dodecylene group, and the like.

In addition, the substituted or unsubstituted aralkylene group used in, for example, L₁ to L₃ as the divalent connecting group may be represented by —(CH₂)ₓ—Ar′—, —Ar′—(CH₂)ₓ—, or —(CH₂)ₓ—Ar′—(CH₂)_{y}—, where Ar′ may represent an arylene group having 6 to 18 carbon atoms for forming a ring (such as a phenylene group, a naphthylene group, and/or the like), and each of x and y may represent an integer selected from 1 to 24. In the arylene group, the total number of carbon atoms represented by x, y and Ar′ may be from 7 to 20, and in some embodiments may be from 7 to 14, but is not limited thereto.

Non-limiting examples of the substituted or unsubstituted arylene group used in, for example, L₁ to L₃ as the divalent connecting group may include a phenylene group, a biphenylene group, a terphenylene group, a naphthylene group, an anthracenylene group, a fluorenylene group, a triphenylene group, and the like. In one embodiment, L₁ to L₃ may each independently be the substituted or unsubstituted phenylene group.

In addition, non-limiting examples of the substituted or unsubstituted heteroarylene group used in, for example, L₁ to L₃ as the divalent connecting group may include a pyridylene group, a dibenzofurylene group, a dibenzoethylylene group, and the like.

Non-limiting examples of the substituted or unsubstituted silyl group used in, for example, L₁ to L₃ as the divalent connecting group may include a dimethylsilylene group and a diphenylsilylene group.

As described above, in a material for an organic electroluminescent device according to embodiments of the present disclosure, an azacarbazole part (e.g., azacarbazole moiety) is introduced (e.g., coupled) to a meta position of a phenylene group which is bonded, directly or via a connecting group (for example, L₁ in Formula 1), with the nitrogen atom (N) of an amine moiety, thereby improving the degree of amorphousness of the material as well as increasing the carrier mobility. The amine moiety may be used to increase the emission lifetime. In addition, the azacarbazole part (e.g., azacarbazole moiety) may be used to further increase the durability, electron resistance, and hence emission lifetime of the material for an organic electroluminescent device.

According to one or more example embodiments of the present disclosure, a material for an organic electroluminescent device may be represented by Formula 1, in which Ar₁ and Ar₂ may be each independently a substituted or unsubstituted phenyl group, and L₁ to L₃ may be each independently a substituted or unsubstituted phenylene group. According to an example embodiment of the present disclosure, a material for an organic electroluminescent device may be an amine compound represented by the following Formula 2:

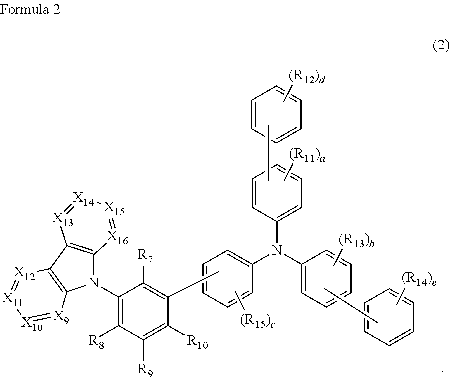

According to one or more embodiments of the present disclosure, a material for an organic electroluminescent device may be represented by Formula 2, wherein X₉ to X₁₆ may be each independently selected from N and CR₆, and at least one of X₉ to X₁₆ may be N. In one embodiment, only one of X₉ to X₁₆ is N, but X₉ to X₁₆ are not limited thereto. R₆ to R₁₅ may be each independently selected from a substituted or unsubstituted aryl group having 6 to 30 carbon atoms for forming a ring, a substituted or unsubstituted heteroaryl group having 1 to 30 carbon atoms for forming a ring, an alkyl group having 1 to 15 carbon atoms, a substituted or unsubstituted silyl group, a halogen atom, hydrogen, and deuterium.

The groups used in R₆ to R₁₅ in Formula 2, for example, a substituted or unsubstituted aryl group having 6 to 30 carbon atoms for forming a ring, a substituted or unsubstituted heteroaryl group having 1 to 30 carbon atoms for forming a ring, an alkyl group having 1 to 15 carbon atoms, a silyl group, and/or the halogen atom, may be respectively the same as the substituted or unsubstituted aryl group having 6 to 30 carbon atoms for forming a ring, the substituted or unsubstituted heteroaryl group having 1 to 30 carbon atoms for forming a ring, the alkyl group having 1 to 15 carbon atoms, the substituted or unsubstituted silyl group and the halogen atom used in R₁ to R₅ in Formula 1.

In Formula 2, respective atoms in a plurality of adjacent R₆, respective atoms in a plurality of adjacent R₈ to R₁₀, respective atoms in a plurality of adjacent R₁₁, respective atoms in a plurality of adjacent R₁₂, respective atoms in a plurality of adjacent R₁₃, respective atoms in a plurality of adjacent R₁₄ and/or respective atoms in a plurality of adjacent R₁₅ may be bonded (e.g., coupled) to each other to form, for example, a saturated or unsaturated ring.

According to some embodiments of the present disclosure, a material for an organic electroluminescent device may be represented by Formula 1, in which Ar₁ and Ar₂ may each include a substituted or unsubstituted phenyl group, and L₁ to L₃ may each include a substituted or unsubstituted phenylene group, and a conjugated amine thus obtained may enhance the charge resistance of the material.

According to one or more embodiments of the present disclosure, a material for an organic electroluminescent device may include at least one selected from Compounds 1 to 60.

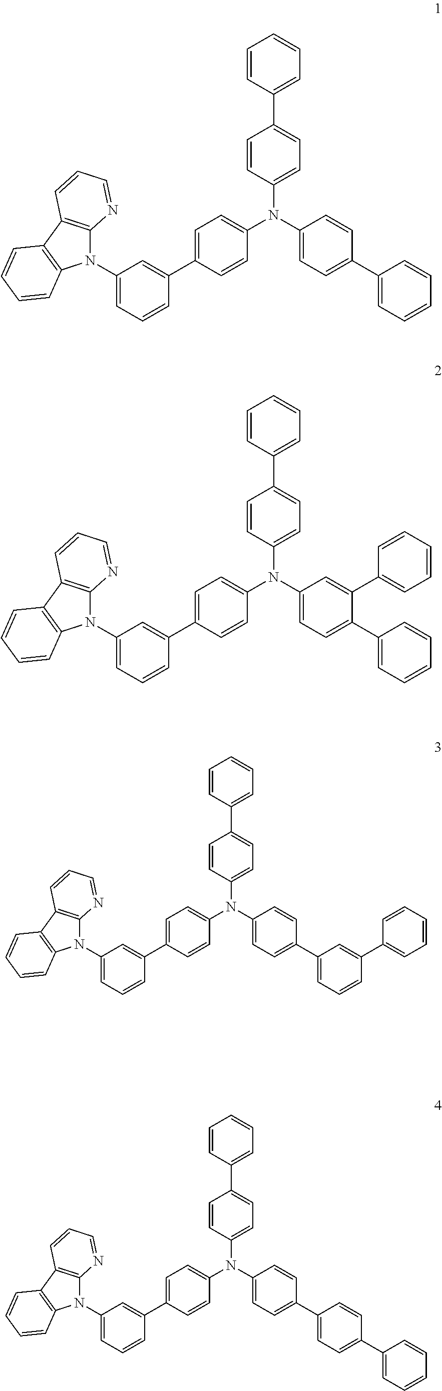 [SMILES: c1ccc(-c2ccc(N(c3ccc(-c4ccccc4)cc3)c3ccc(-c4cccc(-n5c6ccccc6c6cccnc65)c4)cc3)cc2)cc1 ; c1ccc(-c2ccc(N(c3ccc(-c4cccc(-n5c6ccccc6c6cccnc65)c4)cc3)c3ccc(-c4ccccc4)c(-c4ccccc4)c3)cc2)cc1 ; c1ccc(-c2ccc(N(c3ccc(-c4cccc(-c5ccccc5)c4)cc3)c3ccc(-c4cccc(-n5c6ccccc6c6cccnc65)c4)cc3)cc2)cc1 ; c1ccc(-c2ccc(-c3ccc(N(c4ccc(-c5ccccc5)cc4)c4ccc(-c5cccc(-n6c7ccccc7c7cccnc76)c5)cc4)cc3)cc2)cc1]

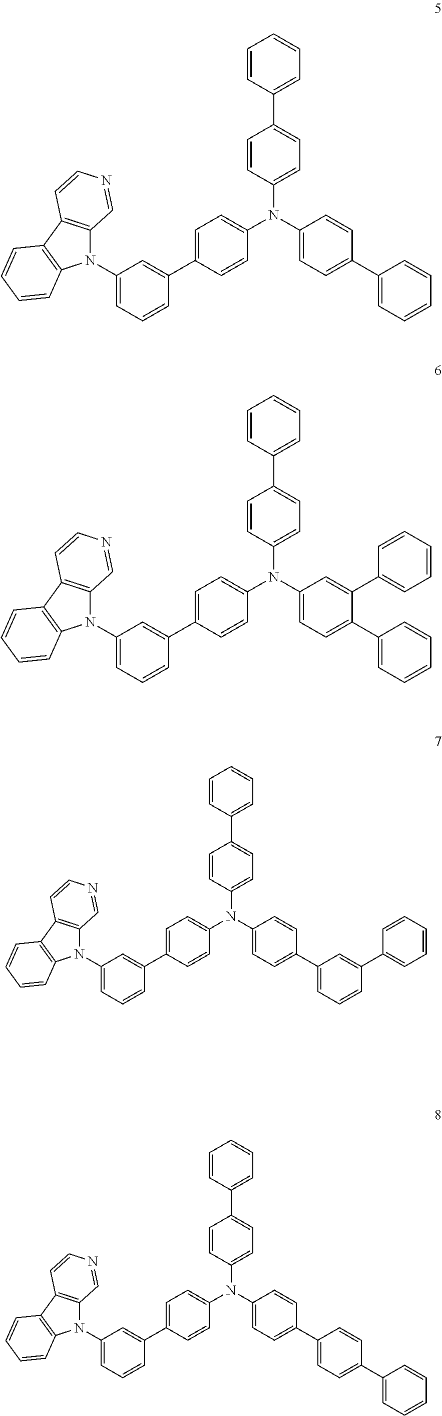 [SMILES: c1ccc(-c2ccc(N(c3ccc(-c4ccccc4)cc3)c3ccc(-c4cccc(-n5c6ccccc6c6ccncc65)c4)cc3)cc2)cc1 ; c1ccc(-c2ccc(N(c3ccc(-c4cccc(-n5c6ccccc6c6ccncc65)c4)cc3)c3ccc(-c4ccccc4)c(-c4ccccc4)c3)cc2)cc1 ; c1ccc(-c2ccc(N(c3ccc(-c4cccc(-c5ccccc5)c4)cc3)c3ccc(-c4cccc(-n5c6ccccc6c6ccncc65)c4)cc3)cc2)cc1 ; c1ccc(-c2ccc(-c3ccc(N(c4ccc(-c5ccccc5)cc4)c4ccc(-c5cccc(-n6c7ccccc7c7ccncc76)c5)cc4)cc3)cc2)cc1]

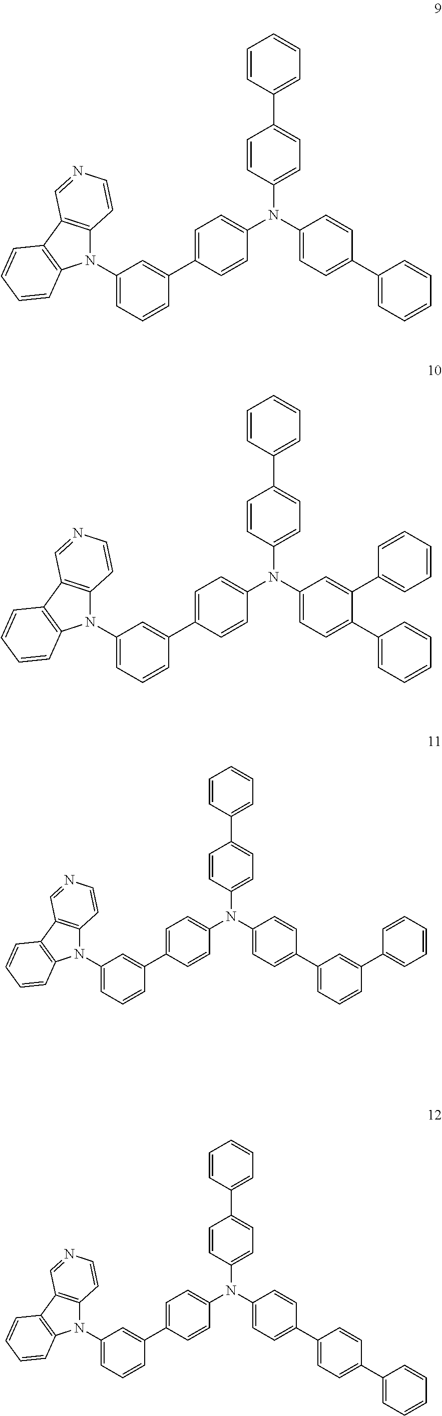 [SMILES: c1ccc(-c2ccc(N(c3ccc(-c4ccccc4)cc3)c3ccc(-c4cccc(-n5c6ccccc6c6cnccc65)c4)cc3)cc2)cc1 ; c1ccc(-c2ccc(N(c3ccc(-c4cccc(-n5c6ccccc6c6cnccc65)c4)cc3)c3ccc(-c4ccccc4)c(-c4ccccc4)c3)cc2)cc1 ; c1ccc(-c2ccc(N(c3ccc(-c4cccc(-c5ccccc5)c4)cc3)c3ccc(-c4cccc(-n5c6ccccc6c6cnccc65)c4)cc3)cc2)cc1 ; c1ccc(-c2ccc(-c3ccc(N(c4ccc(-c5ccccc5)cc4)c4ccc(-c5cccc(-n6c7ccccc7c7cnccc76)c5)cc4)cc3)cc2)cc1]

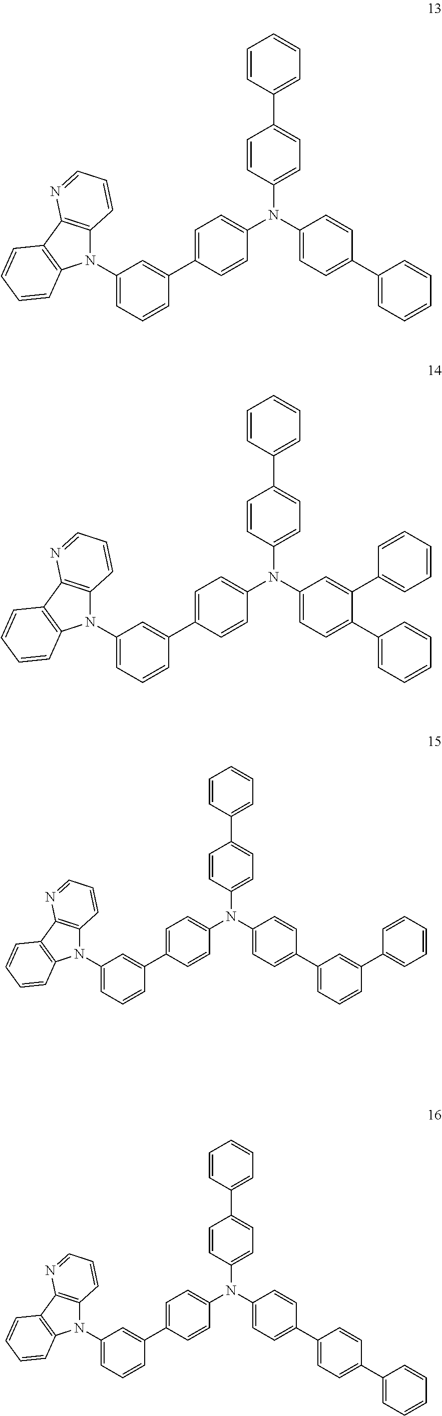 [SMILES: c1ccc(-c2ccc(N(c3ccc(-c4ccccc4)cc3)c3ccc(-c4cccc(-n5c6ccccc6c6ncccc65)c4)cc3)cc2)cc1 ; c1ccc(-c2ccc(N(c3ccc(-c4cccc(-n5c6ccccc6c6ncccc65)c4)cc3)c3ccc(-c4ccccc4)c(-c4ccccc4)c3)cc2)cc1 ; c1ccc(-c2ccc(N(c3ccc(-c4cccc(-c5ccccc5)c4)cc3)c3ccc(-c4cccc(-n5c6ccccc6c6ncccc65)c4)cc3)cc2)cc1 ; c1ccc(-c2ccc(-c3ccc(N(c4ccc(-c5ccccc5)cc4)c4ccc(-c5cccc(-n6c7ccccc7c7ncccc76)c5)cc4)cc3)cc2)cc1]

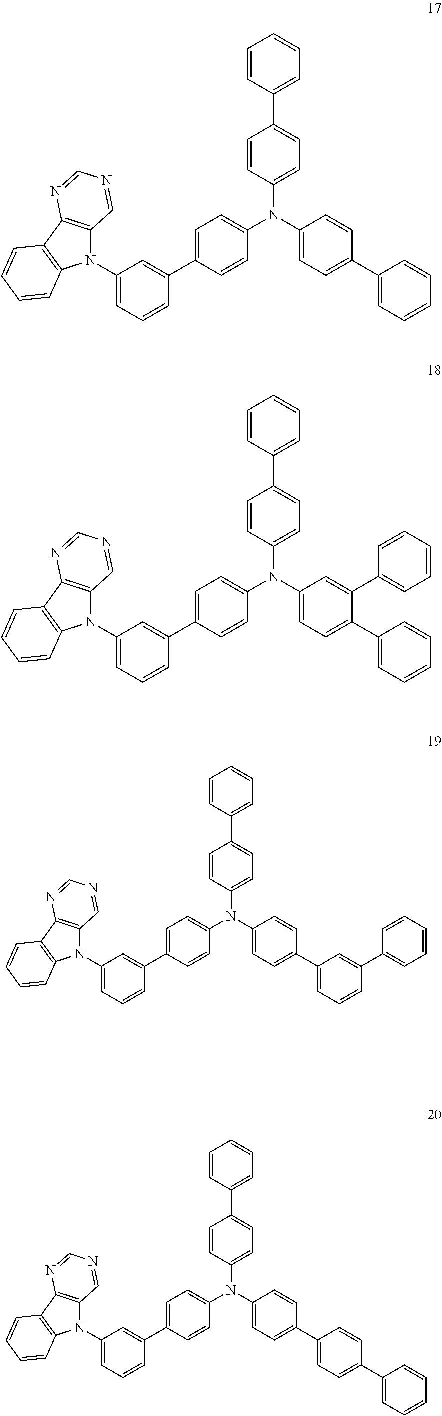 [SMILES: c1ccc(-c2ccc(N(c3ccc(-c4ccccc4)cc3)c3ccc(-c4cccc(-n5c6ccccc6c6ncncc65)c4)cc3)cc2)cc1 ; c1ccc(-c2ccc(N(c3ccc(-c4cccc(-n5c6ccccc6c6ncncc65)c4)cc3)c3ccc(-c4ccccc4)c(-c4ccccc4)c3)cc2)cc1 ; c1ccc(-c2ccc(N(c3ccc(-c4cccc(-c5ccccc5)c4)cc3)c3ccc(-c4cccc(-n5c6ccccc6c6ncncc65)c4)cc3)cc2)cc1 ; c1ccc(-c2ccc(-c3ccc(N(c4ccc(-c5ccccc5)cc4)c4ccc(-c5cccc(-n6c7ccccc7c7ncncc76)c5)cc4)cc3)cc2)cc1]

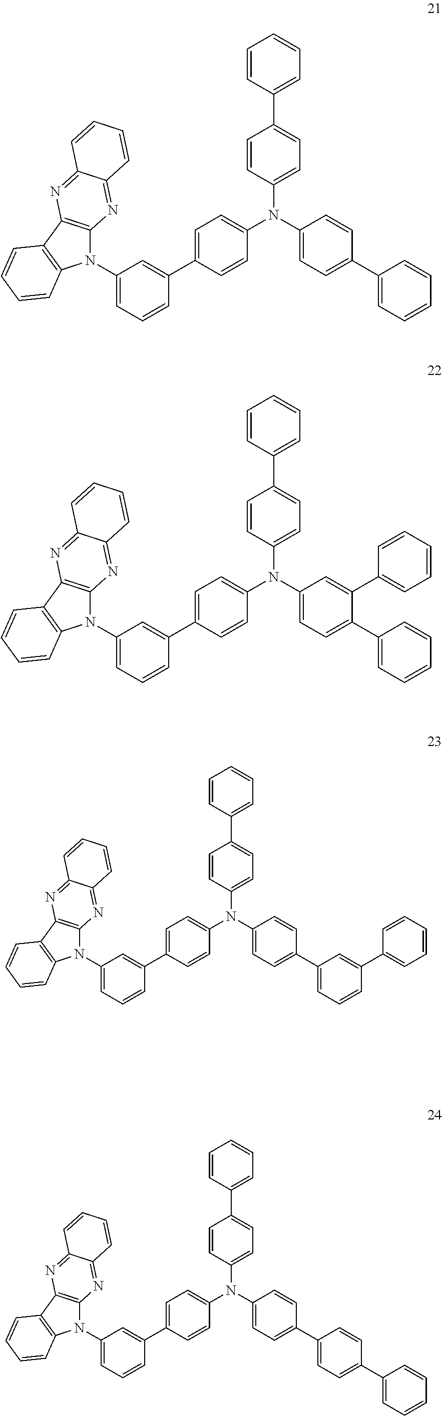 [SMILES: c1ccc(-c2ccc(N(c3ccc(-c4ccccc4)cc3)c3ccc(-c4cccc(-n5c6ccccc6c6nc7ccccc7nc65)c4)cc3)cc2)cc1 ; c1ccc(-c2ccc(N(c3ccc(-c4cccc(-n5c6ccccc6c6nc7ccccc7nc65)c4)cc3)c3ccc(-c4ccccc4)c(-c4ccccc4)c3)cc2)cc1 ; c1ccc(-c2ccc(N(c3ccc(-c4cccc(-c5ccccc5)c4)cc3)c3ccc(-c4cccc(-n5c6ccccc6c6nc7ccccc7nc65)c4)cc3)cc2)cc1 ; c1ccc(-c2ccc(-c3ccc(N(c4ccc(-c5ccccc5)cc4)c4ccc(-c5cccc(-n6c7ccccc7c7nc8ccccc8nc76)c5)cc4)cc3)cc2)cc1]

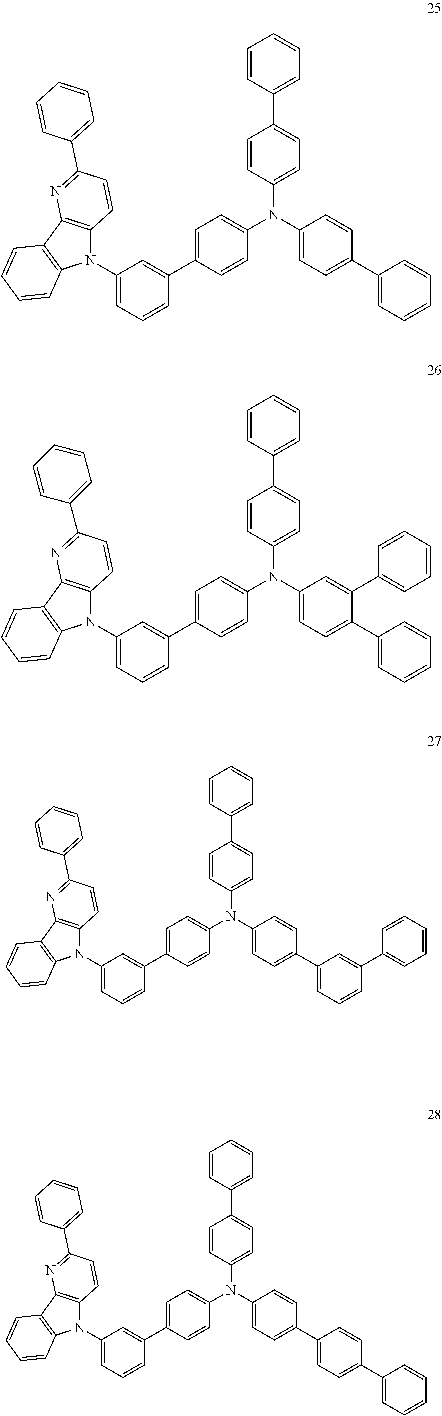 [SMILES: c1ccc(-c2ccc(N(c3ccc(-c4ccccc4)cc3)c3ccc(-c4cccc(-n5c6ccccc6c6nc(-c7ccccc7)ccc65)c4)cc3)cc2)cc1 ; c1ccc(-c2ccc(N(c3ccc(-c4cccc(-n5c6ccccc6c6nc(-c7ccccc7)ccc65)c4)cc3)c3ccc(-c4ccccc4)c(-c4ccccc4)c3)cc2)cc1 ; c1ccc(-c2ccc(N(c3ccc(-c4cccc(-c5ccccc5)c4)cc3)c3ccc(-c4cccc(-n5c6ccccc6c6nc(-c7ccccc7)ccc65)c4)cc3)cc2)cc1 ; c1ccc(-c2ccc(-c3ccc(N(c4ccc(-c5ccccc5)cc4)c4ccc(-c5cccc(-n6c7ccccc7c7nc(-c8ccccc8)ccc76)c5)cc4)cc3)cc2)cc1]

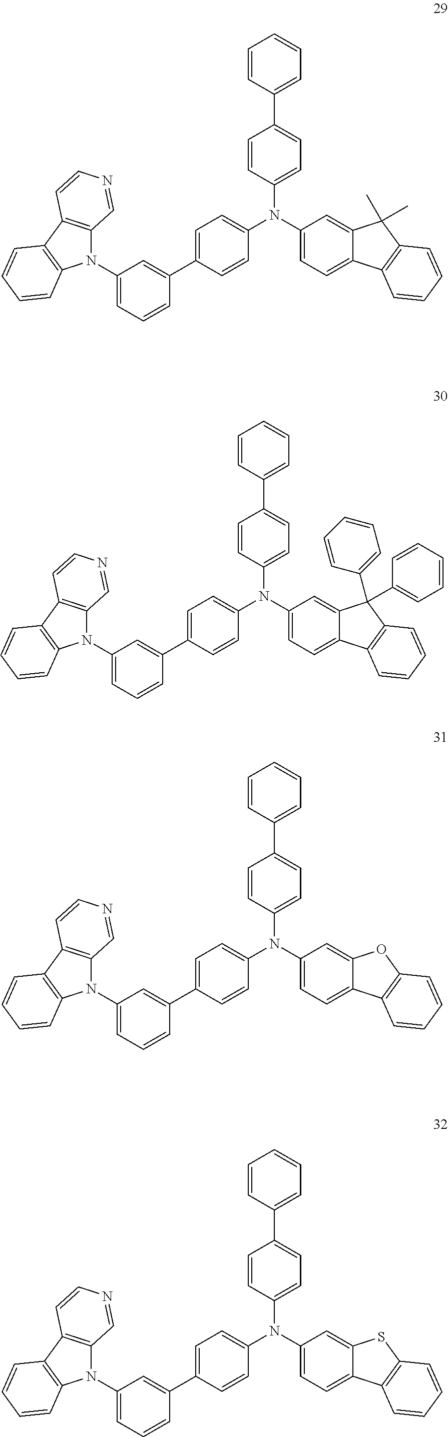 [SMILES: CC1(C)c2ccccc2-c2ccc(N(c3ccc(-c4ccccc4)cc3)c3ccc(-c4cccc(-n5c6ccccc6c6ccncc65)c4)cc3)cc21 ; c1ccc(-c2ccc(N(c3ccc(-c4cccc(-n5c6ccccc6c6ccncc65)c4)cc3)c3ccc4c(c3)C(c3ccccc3)(c3ccccc3)c3ccccc3-4)cc2)cc1 ; c1ccc(-c2ccc(N(c3ccc(-c4cccc(-n5c6ccccc6c6ccncc65)c4)cc3)c3ccc4c(c3)oc3ccccc34)cc2)cc1 ; c1ccc(-c2ccc(N(c3ccc(-c4cccc(-n5c6ccccc6c6ccncc65)c4)cc3)c3ccc4c(c3)sc3ccccc34)cc2)cc1]

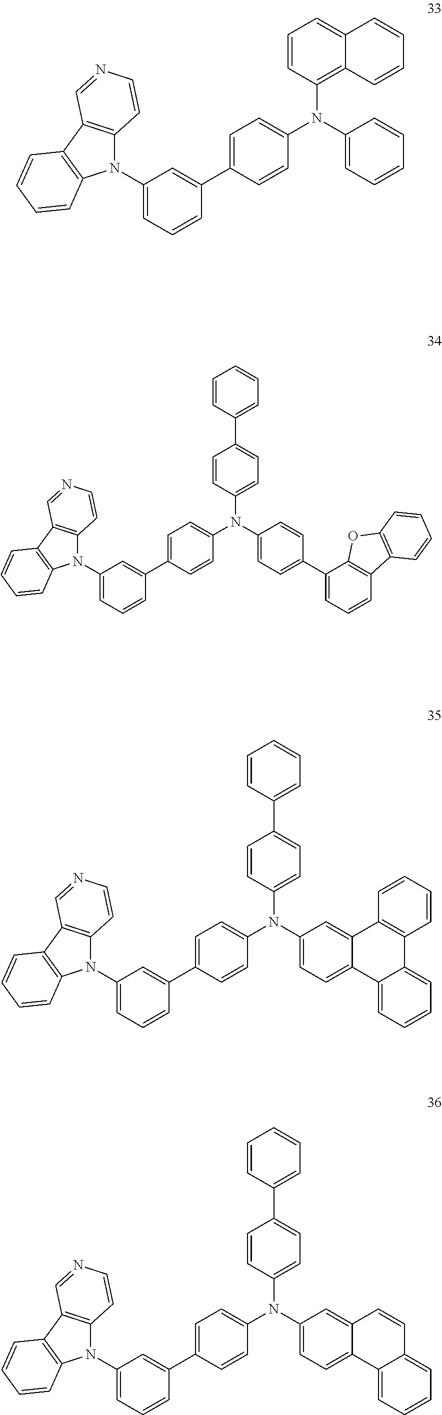 [SMILES: c1ccc(N(c2ccc(-c3cccc(-n4c5ccccc5c5cnccc54)c3)cc2)c2cccc3ccccc23)cc1 ; c1ccc(-c2ccc(N(c3ccc(-c4cccc(-n5c6ccccc6c6cnccc65)c4)cc3)c3ccc(-c4cccc5c4oc4ccccc45)cc3)cc2)cc1 ; c1ccc(-c2ccc(N(c3ccc(-c4cccc(-n5c6ccccc6c6cnccc65)c4)cc3)c3ccc4c5ccccc5c5ccccc5c4c3)cc2)cc1 ; c1ccc(-c2ccc(N(c3ccc(-c4cccc(-n5c6ccccc6c6cnccc65)c4)cc3)c3ccc4c(ccc5ccccc54)c3)cc2)cc1]

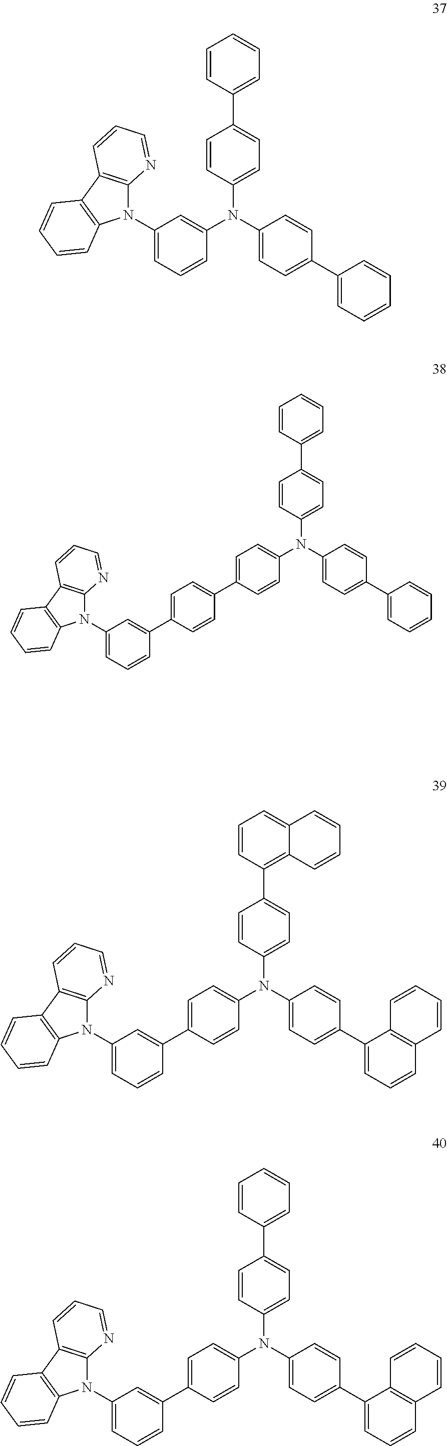 [SMILES: c1ccc(-c2ccc(N(c3ccc(-c4ccccc4)cc3)c3cccc(-n4c5ccccc5c5cccnc54)c3)cc2)cc1 ; c1ccc(-c2ccc(N(c3ccc(-c4ccccc4)cc3)c3ccc(-c4ccc(-c5cccc(-n6c7ccccc7c7cccnc76)c5)cc4)cc3)cc2)cc1 ; c1cc(-c2ccc(N(c3ccc(-c4cccc5ccccc45)cc3)c3ccc(-c4cccc5ccccc45)cc3)cc2)cc(-n2c3ccccc3c3cccnc32)c1 ; c1ccc(-c2ccc(N(c3ccc(-c4cccc(-n5c6ccccc6c6cccnc65)c4)cc3)c3ccc(-c4cccc5ccccc45)cc3)cc2)cc1]

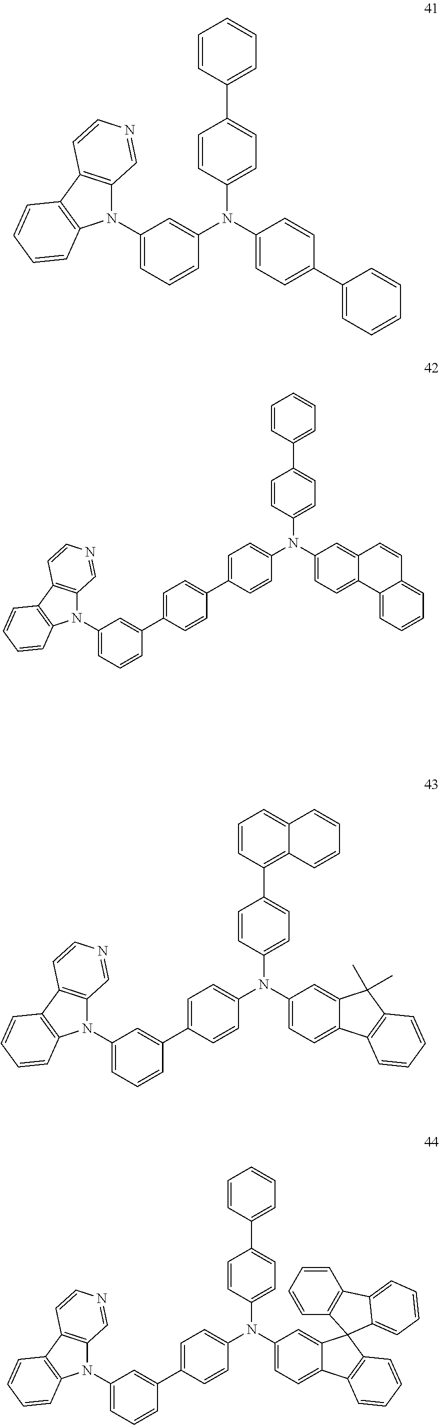 [SMILES: c1ccc(-c2ccc(N(c3ccc(-c4ccccc4)cc3)c3cccc(-n4c5ccccc5c5ccncc54)c3)cc2)cc1 ; c1ccc(-c2ccc(N(c3ccc(-c4ccc(-c5cccc(-n6c7ccccc7c7ccncc76)c5)cc4)cc3)c3ccc4c(ccc5ccccc54)c3)cc2)cc1 ; CC1(C)c2ccccc2-c2ccc(N(c3ccc(-c4cccc(-n5c6ccccc6c6ccncc65)c4)cc3)c3ccc(-c4cccc5ccccc45)cc3)cc21 ; c1ccc(-c2ccc(N(c3ccc(-c4cccc(-n5c6ccccc6c6ccncc65)c4)cc3)c3ccc4c(c3)C3(c5ccccc5-c5ccccc53)c3ccccc3-4)cc2)cc1]

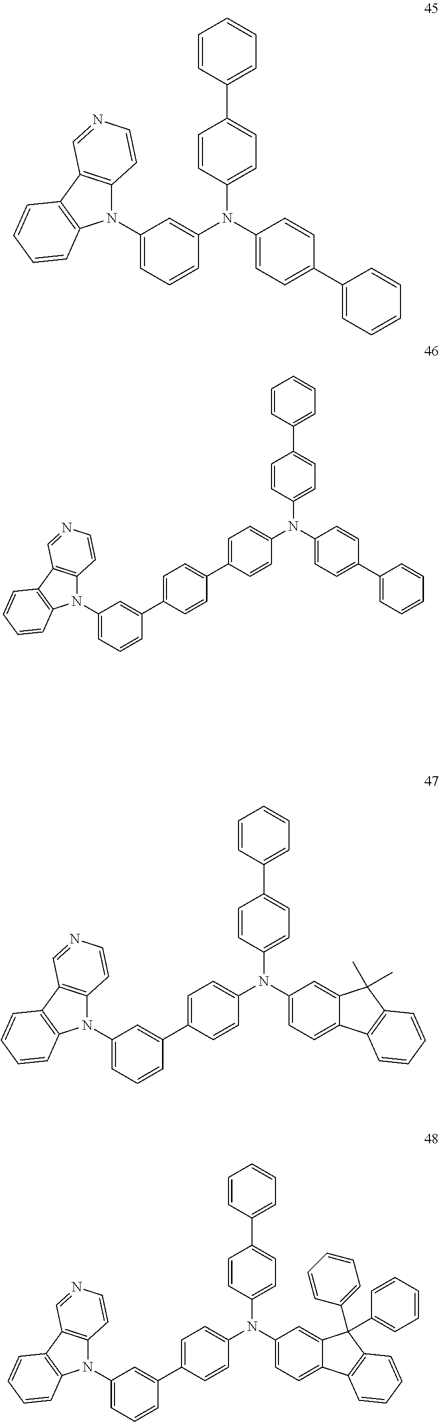 [SMILES: c1ccc(-c2ccc(N(c3ccc(-c4ccccc4)cc3)c3cccc(-n4c5ccccc5c5cnccc54)c3)cc2)cc1 ; c1ccc(-c2ccc(N(c3ccc(-c4ccccc4)cc3)c3ccc(-c4ccc(-c5cccc(-n6c7ccccc7c7cnccc76)c5)cc4)cc3)cc2)cc1 ; CC1(C)c2ccccc2-c2ccc(N(c3ccc(-c4ccccc4)cc3)c3ccc(-c4cccc(-n5c6ccccc6c6cnccc65)c4)cc3)cc21 ; c1ccc(-c2ccc(N(c3ccc(-c4cccc(-n5c6ccccc6c6cnccc65)c4)cc3)c3ccc4c(c3)C(c3ccccc3)(c3ccccc3)c3ccccc3-4)cc2)cc1]

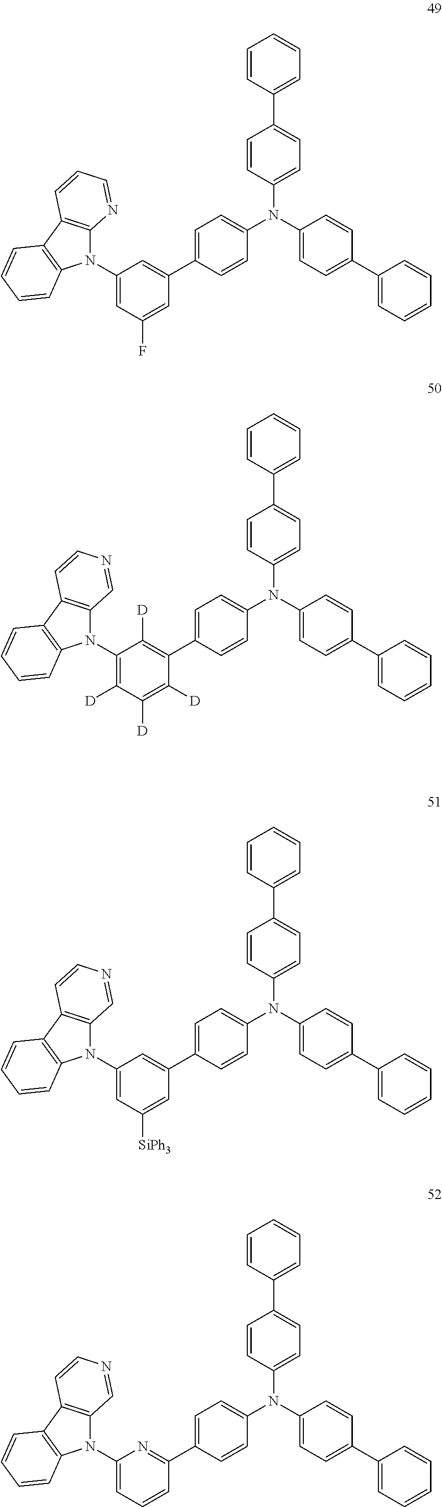

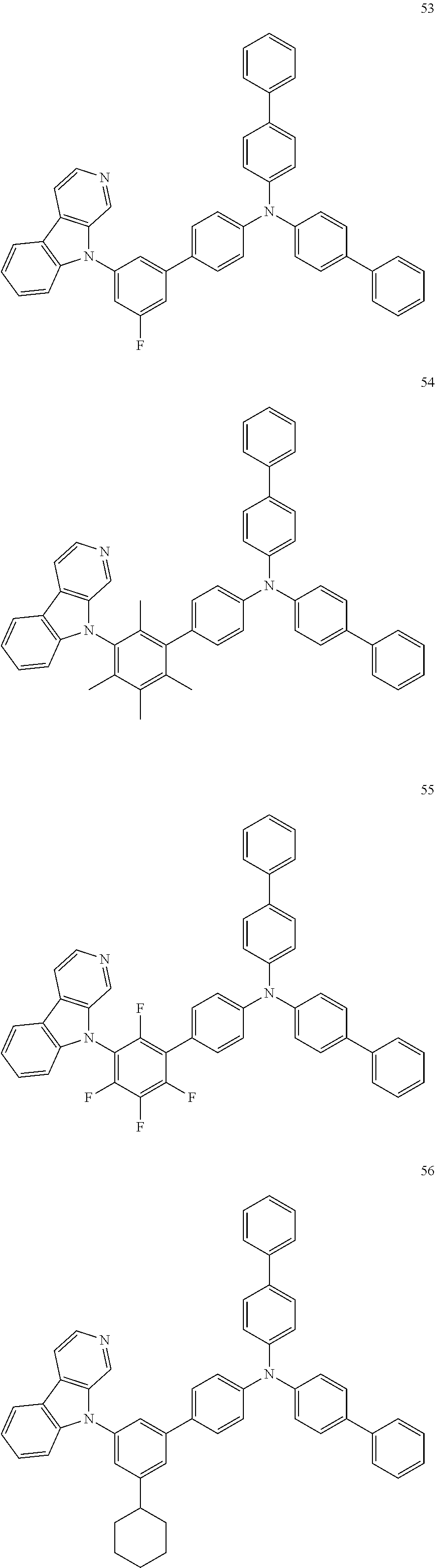 [SMILES: Fc1cc(-c2ccc(N(c3ccc(-c4ccccc4)cc3)c3ccc(-c4ccccc4)cc3)cc2)cc(-n2c3ccccc3c3ccncc32)c1 ; Cc1c(C)c(-c2ccc(N(c3ccc(-c4ccccc4)cc3)c3ccc(-c4ccccc4)cc3)cc2)c(C)c(-n2c3ccccc3c3ccncc32)c1C ; Fc1c(F)c(-c2ccc(N(c3ccc(-c4ccccc4)cc3)c3ccc(-c4ccccc4)cc3)cc2)c(F)c(-n2c3ccccc3c3ccncc32)c1F ; c1ccc(-c2ccc(N(c3ccc(-c4ccccc4)cc3)c3ccc(-c4cc(C5CCCCC5)cc(-n5c6ccccc6c6ccncc65)c4)cc3)cc2)cc1]

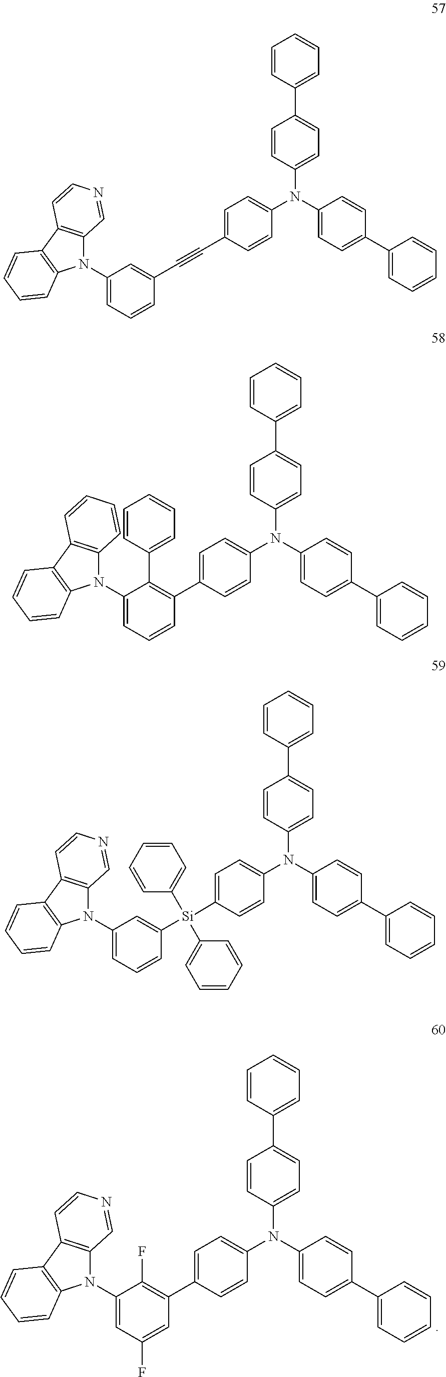 [SMILES: C(#Cc1cccc(-n2c3ccccc3c3ccncc32)c1)c1ccc(N(c2ccc(-c3ccccc3)cc2)c2ccc(-c3ccccc3)cc2)cc1 ; c1ccc(-c2ccc(N(c3ccc(-c4ccccc4)cc3)c3ccc(-c4cccc(-n5c6ccccc6c6ccccc65)c4-c4ccccc4)cc3)cc2)cc1 ; c1ccc(-c2ccc(N(c3ccc(-c4ccccc4)cc3)c3ccc([Si](c4ccccc4)(c4ccccc4)c4cccc(-n5c6ccccc6c6ccncc65)c4)cc3)cc2)cc1 ; Fc1cc(-c2ccc(N(c3ccc(-c4ccccc4)cc3)c3ccc(-c4ccccc4)cc3)cc2)c(F)c(-n2c3ccccc3c3ccncc32)c1]

According to one or more embodiments of the present disclosure, a material for an organic electroluminescent device may be included in at least one lamination layer which is positioned between an emission layer and an anode. The degree of amorphousness of the material may be improved so that the carrier mobility is increased, and thus, a long lifetime of the organic electroluminescent device may be realized. Furthermore, since the electron resistance of the material is improved, the durability of the material may be improved and a further increase of the lifetime of the organic electroluminescent device may be realized.

According to one or more embodiments of the present disclosure, a lamination layer that includes the material for an organic electroluminescent device is positioned between the emission layer and an anode and may be more adjacent to an emission layer than the other lamination layers. In some embodiments, the layer including the material for an organic electroluminescent device according to embodiments of the present disclosure may be adjacent to the emission layer, and thus deterioration of a layer that is positioned between the anode and the layer including the material for an organic electroluminescent device may be prevented or reduced. The lamination layer may thus be effectively protected from deterioration.

### (Organic Electroluminescent Device)

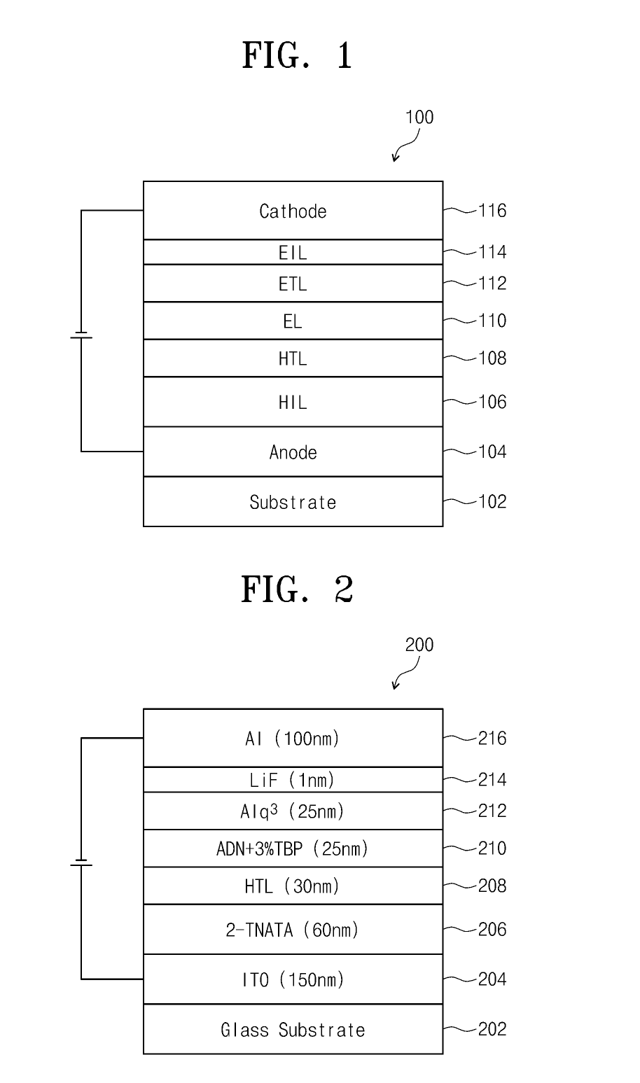

An organic electroluminescent device using the material for an organic electroluminescent device according to one or more embodiments of the present disclosure will now be described. FIG. 1 is a schematic diagram illustrating an organic electroluminescent device 100 according to an embodiment of the present disclosure. The organic electroluminescent device 100 may include, for example, a substrate 102, an anode 104, a hole injection layer 106, a hole transport layer 108, an emission layer 110, an electron transport layer 112, an electron injection layer 114 and a cathode 116. The material for an organic electroluminescent device according to one or more embodiments of the present disclosure may be used in at least one of any of the lamination layers positioned between the emission layer and the anode.

Herein, a description will be given of an example embodiment in which the material for an organic electroluminescent device is used in the hole transport layer 108.

The substrate 102 may include a transparent glass substrate, a semiconductor substrate made of silicon, or a flexible substrate made of resin, and/or the like.

The anode 104 may be positioned on the substrate 102 and may include indium tin oxide (ITO), indium zinc oxide (IZO), and/or the like.

The hole injection layer (HIL) 106 may include any suitable material and may be formed to a thickness of about 10 nm to about 150 nm. Non-limiting examples of this material include triphenylamine-containing poly ether ketone (TPAPEK), 4-isopropyl-4′-methyldiphenyliodoniumtetrakis(pentafluorophenyl)borate (PPBI), N,N′-diphenyl-N,N′-bis-[4-(phenyl-m-trile-amino)-phenyl]-biphenyl-4,4′-diamine (DNTPD), a phthalocyanine compound such as copper phthalocyanine, 4,4′,4″-tris(3-methylphenylphenylamino)triphenylamine (m-MTDATA), N,N′-di(1-naphthyl)-N,N′-diphenylbenzidine (NPB), 4,4′,4″-tris{N,N-diphenylamino}triphenylamine (TDATA), 4,4′,4″-tris(N,N-2-naphthylphenylamino)triphenylamine (2-TNATA), polyaniline/dodecylbenzenesulfonic acid (PANI/DBSA), poly(3,4-ethylenedioxythiophene)/poly(4-styrenesulfonate (PEDOT/PSS), polyaniline/camphorsulfonic acid (PANI/CSA), polyaniline/poly(4-styrenesulfonate (PANI/PSS), and the like.

The hole transport layer (HTL) 108 may be formed on the hole injection layer 106 using the material for an organic electroluminescent device according to embodiments of the present disclosure. In some embodiments, the HTL may have a thickness of about 10 nm to about 150 nm. In some embodiments, the hole transport layer 108 including the material for an organic electroluminescent device may be formed through vacuum evaporation.

The emission layer (EL) 110 may be formed on the hole transport layer 108 using any suitable host material to a thickness of about 10 nm to about 60 nm. The host material used in the emission layer 110 may include, for example, tris(8-quinolinolato)aluminum (Alq3), 4,4′-N,N′-dicarbazole-biphenyl (CBP), poly(N-vinylcarbazole) (PVK), 9,10-di(naphthalene-2-yl)anthracene (ADN), 4,4′,4″-tris(N-carbazolyl)triphenylamine (TCTA), 1,3,5-tris(N-phenylbenzimidazole-2-yl)benzene (TPBI), 3-tert-butyl-9,10-di(naphtho-2-yl)anthracene (TBADN), distyrylarylene (DSA), 4,4′-bis(9-carbazole)-2,2′-dimethyl-biphenyl (dmCBP), and/or the like.

The emission layer 110 may include but is not limited to the following dopant materials: styryl derivatives (such as 1,4-bis[2-(3-N-ethylcarbazolyl)vinyl]benzene (BCzVB), 4-(di-p-tolylamino)-4′-[(di-p-tolylamino)styryl]stilbene (DPAVB), N-(4-((E)-2-(6-((E)-4-(diphenylamino)styryl)naphthalene-2-yl)vinyl)phenyl-N-phenylbenzeneamine (N-BDAVBI)), perylene and/or derivatives thereof (such as 2,5,8,11-tetra-t-butylperylene (TBPe)), pyrene and/or derivatives thereof (such as 1,1-dipyrene, 1,4-dipyrenylbenzene, 1,4-bis(N,N-diphenylamino)pyrene), and/or the like.

The electron transport layer (ETL) 112 may be formed on the emission layer 110 to a thickness of about 15 nm to about 50 nm using, for example, tris(8-hydroxyquinolinato)aluminum (Alq3) or a material having a nitrogen-containing aromatic ring (for example, a material including a pyridine ring such as 1,3,5-tri[(3-pyridyl)-phen-3-yl]benzene, a material including a triazine ring such as 2,4,6-tris(3′-(pyridine-3-yl)biphenyl-3-yl)1,3,5-triazine, and/or a material including an imidazole derivative such as 2-(4-N-Phenylbenzoimidazolyl-1-ylphenyl)-9,10-dinaphthylanthracene).

The electron injection layer (EIL) 114 may be formed on the electron transport layer 112 to a thickness of about 0.3 nm to about 9 nm using a material including, for example, lithium fluoride (LiF), lithium-8-quinolinato (Liq), and/or the like.

The cathode 116 may be positioned on the electron injection layer 114 and may be formed using a metal such as aluminum (Al), silver (Ag), lithium (Li), magnesium (Mg) and/or calcium (Ca), and/or a transparent material such as ITO and/or IZO.

In one or more embodiments, each electrode and each layer constituting the organic electroluminescent device as described above may be formed by one or more suitable layer forming methods such as vacuum evaporation, sputtering, and/or other various coating methods, depending on the material for forming each electrode or layer. In some embodiments, the hole transport layer 108 including the material for an organic electroluminescent device according to embodiments of the present disclosure may be formed through vacuum evaporation.

In the organic electroluminescent device 100 according to embodiments of the present disclosure, a hole transport layer capable of realizing a long lifetime and high efficiency may include the material for an organic electroluminescent device.

One or more embodiments of the present disclosure relate to use of the presently described material for an organic electroluminescent device in the hole injection layer. In one or more embodiments, an organic electroluminescent device with a long lifetime may be manufactured by using the material for an organic electroluminescent device in at least one of the lamination layers positioned between the emission layer and the anode.

In one of more embodiments of the present disclosure, the material for an organic electroluminescent device may be applied to an active matrix type organic electroluminescent device using a thin film transistor (TFT).

### (Manufacturing Method)

According to one or more embodiments of the present disclosure, a material for an organic electroluminescent device may be synthesized, for example, as follows.

### (Method of Synthesizing Compound 5 Shown Below)

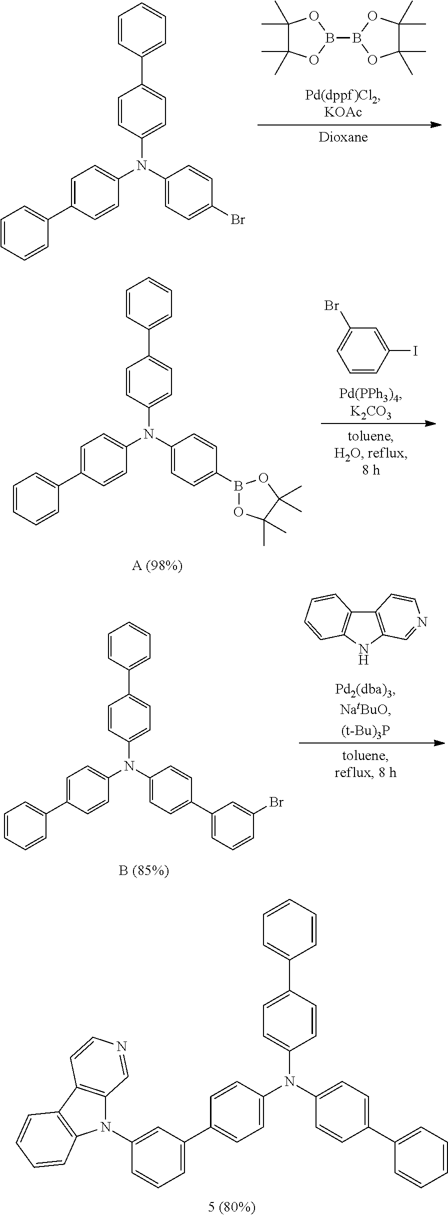

### (Synthesis of Compound A)

Under an Ar atmosphere, 53.8 g of N-[1,1′-biphenyl]-4-yl-N-(4-bromophenyl)-[1,1′-biphenyl]-4-amine, 6.46 g of Pd(dppf)Cl₂.CH₂Cl₂, 33.3 g of KOAc and 33.0 g of bis(pinacolato)diboron were added to a 2 L flask, followed by degassing under vacuum and stirring the resulting mixture in a dioxane solvent at about 100° C. for about 12 hours. Thereafter, the solvent was distilled, CH₂Cl₂ and water were added to the remaining material, an organic phase was separated, and magnesium sulfate and activated clay were added thereto. Suction filtration of the organic phase was performed, and solvent was distilled. The crude product thus obtained was purified by silica gel column chromatography (using a solvent mixture of dichloromethane and hexane at a ratio of 1:3) to produce 58.0 g (Yield 98%) of Compound A as a white solid. The molecular weight of Compound A measured by Fast Atom Bombardment Mass Spectrometry (FAB-MS) was 523.

### (Synthesis of Compound B)

Under an Ar atmosphere, 4.0 g of Compound A, 3.24 g of 1-iodo-3-bromobenzene, 0.62 g of Pd(PPh₃)₄ and 2.11 g of potassium carbonate were added to a 500 mL, three-necked flask, followed by heating and stirring in 200 mL of toluene at about 90° C. for about 8 hours. After air cooling, water was added to the reaction, an organic layer was separated, and solvent was distilled. The crude product thus obtained was purified by silica gel column chromatography (using a solvent mixture of dichloromethane and hexane) and recrystallized using a solvent mixture of toluene and hexane to produce 3.59 g (Yield 85%) of Compound B as a white solid.

The molecular weight of Compound B measured by FAB-MS was 552.

### (Synthesis of Compound 5)

Under an Ar atmosphere, 5.30 g of Compound B, 1.78 g of 9H-Pyrido[3,4-b]indole, 0.72 g of tris(dibenzylideneacetone)dipalladium(0) (Pd₂(dba)₃), 255 mg of Tri-tert-butylphosphine (t-Bu)₃P), and 2.89 g of sodium tert-butoxide were added to a 300 mL, three-necked flask, followed by heating and stirring in a solvent of 120 mL toluene at about 90° C. for about 8 hours. After air cooling, water was added to the reaction, an organic layer was separated, and solvent was distilled. The crude product thus obtained was purified by silica gel column chromatography (a solvent mixture of dichloromethane and hexane) and recrystallized using a solvent mixture of toluene and hexane to produce 4.91 g (Yield 80%) of Compound 5 as a white solid. The molecular weight of Compound 5 measured by FAB-MS was 640. In addition, the chemical shift values (δ) of Compound 5 measured by ¹H-NMR (CDCl₃) were 8.87 (s, 1H), 8.55 (d, 1H, J=7.82 Hz), 8.43 (d, 1H, J=7.60 Hz), 8.09 (s, 1H), 7.94 (d, 1H, J=7.88 Hz), 7.57-7.50 (m, 16H), 7.48-7.41 (m, 6H), 6.69-6.67 (m, 6H).

In addition, material for an organic electroluminescent device according to one or more embodiments of the present disclosure may be synthesized, for example, as follows.

### (Synthesis of Compound 1 Shown Below)

Compound 1 was synthesized through the same (or substantially the same) method as the synthetic method of Compound 5 except for using 9H-Pyrido[2,3-b]indole instead of 9H-Pyrido[3,4-b]indole. The chemical shift values (δ) of Compound 1 measured by ¹H-NMR (CDCl₃) were 8.87 (s, 1H), 8.55 (d, 1H, J=7.82 Hz), 8.43 (d, 1H, J=7.60 Hz), 8.09 (s, 1H), 7.94 (d, 1H, J=7.88 Hz), 7.57-7.50 (m, 16H), 7.48-7.41 (m, 6H), 6.69-6.67 (m, 6H).

### (Synthesis of Compound 9 Shown Below)

Compound 9 was synthesized through the same (or substantially the same) method as the synthetic method of Compound 5 except for using 9H-Pyrido[2,3-b]indole instead of 9H-Pyrido[3,4-b]indole. The chemical shift values (δ) of Compound 9 measured by ¹H-NMR (CDCl₃) were 8.89 (s, 1H), 8.45 (d, 1H, J=7.80 Hz), 8.40 (d, 1H, J=7.90 Hz), 8.09 (s, 1H), 7.94 (d, 1H, J=7.86 Hz), 7.58-7.51 (m, 16H), 7.49-7.43 (m, 6H), 6.76-6.67 (m, 6H).

### (Synthesis of Compound 13 Shown Below)

Compound 13 was synthesized through the same (or substantially the same) method as the synthetic method of Compound 5 except for using 9H-Pyrido[2,3-b]indole instead of 9H-Pyrido[3,4-b]indole. The chemical shift values (δ) of Compound 13 measured by ¹H-NMR (CDCl₃) were 8.66 (s, 1H), 8.50 (d, 1H, J=7.72 Hz), 8.33 (d, 1H, J=7.66 Hz), 8.19 (s, 1H), 7.90 (d, 1H, J=7.88 Hz), 7.61-7.52 (m, 16H), 7.48-7.41 (m, 6H), 6.99-6.77 (m, 6H).

Organic electroluminescent devices of Examples 1 to 4 were manufactured using hole transport material compounds 1, 5, 9, and 13, respectively, and were produced using the above-described manufacturing method.

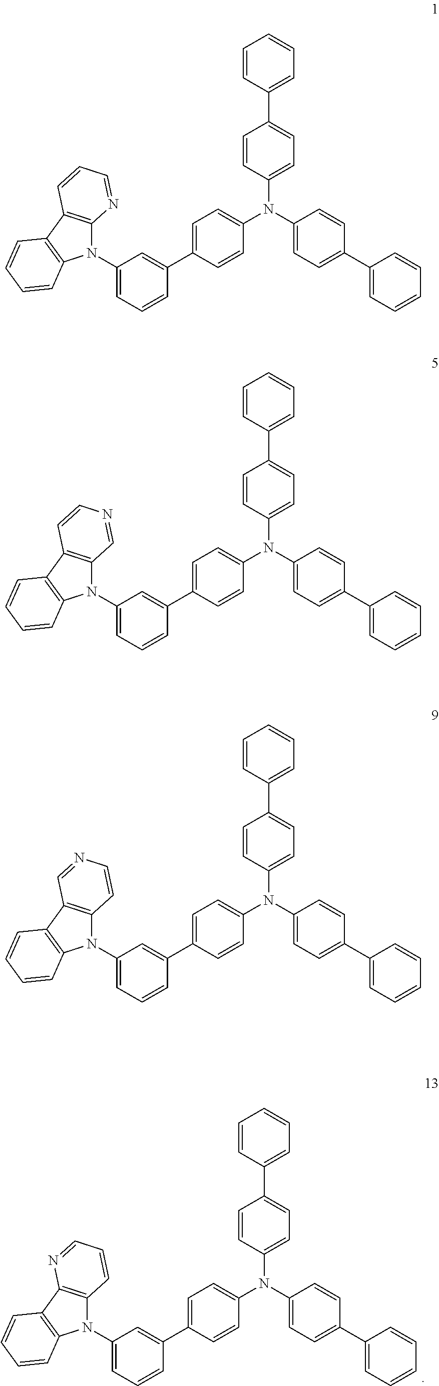 [SMILES: c1ccc(-c2ccc(N(c3ccc(-c4ccccc4)cc3)c3ccc(-c4cccc(-n5c6ccccc6c6cccnc65)c4)cc3)cc2)cc1 ; c1ccc(-c2ccc(N(c3ccc(-c4ccccc4)cc3)c3ccc(-c4cccc(-n5c6ccccc6c6ccncc65)c4)cc3)cc2)cc1 ; c1ccc(-c2ccc(N(c3ccc(-c4ccccc4)cc3)c3ccc(-c4cccc(-n5c6ccccc6c6cnccc65)c4)cc3)cc2)cc1 ; c1ccc(-c2ccc(N(c3ccc(-c4ccccc4)cc3)c3ccc(-c4cccc(-n5c6ccccc6c6ncccc65)c4)cc3)cc2)cc1]

In addition, organic electroluminescent devices of Comparative Examples 1 to 3 were manufactured by respectively using the following Comparative Compounds C1 to C3 as the hole transport materials.

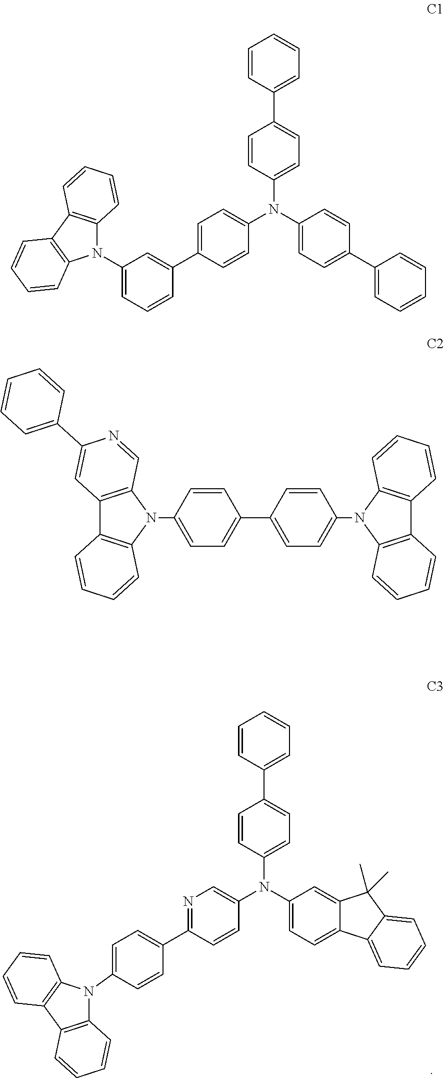 [SMILES: c1ccc(-c2ccc(N(c3ccc(-c4ccccc4)cc3)c3ccc(-c4cccc(-n5c6ccccc6c6ccccc65)c4)cc3)cc2)cc1 ; c1ccc(-c2cc3c4ccccc4n(-c4ccc(-c5ccc(-n6c7ccccc7c7ccccc76)cc5)cc4)c3cn2)cc1 ; CC1(C)c2ccccc2-c2ccc(N(c3ccc(-c4ccccc4)cc3)c3ccc(-c4ccc(-n5c6ccccc6c6ccccc65)cc4)nc3)cc21]

An organic electroluminescent device 200 according to an embodiment of the present disclosure is shown in FIG. 2. In this embodiment, a transparent glass substrate was used as a substrate 202, an anode 204 was formed of ITO to a layer thickness of about 150 nm, a hole injection layer 206 was formed of 2-TNATA to a layer thickness of about 60 nm, a hole transport layer 208 was formed to a layer thickness of about 30 nm and including the material for an organic electroluminescent device according to embodiments of the present disclosure, an emission layer 210 was formed of ADN doped with 3% TBP to a layer thickness of about 25 nm, an electron transport layer 212 was formed of Alq3 to a layer thickness of about 25 nm, an electron injection layer 214 was formed of LiF to a layer thickness of about 1 nm, and a cathode 216 was formed of Al to a layer thickness of about 100 nm.

For the organic electroluminescent device 200 thus manufactured (here, organic electroluminescent devices of Examples 1 to 4 and Comparative Examples 1 to 3), a driving voltage and half life were evaluated. The voltage was obtained at about 10 mA/cm², and the half life was measured as the time necessary for the initial luminance of about 1,000 cd/m² to decrease by 50%. The evaluation results are shown in Table 1.

TABLE 1

| Manufacturing | | Voltage | Half Life |
|---|---|---|---|
| examples of device | Hole transport layer | (V) | LT50 (h) |
| Example 1 | Compound 1 | 6.2 | 1,850 |
| Example 2 | Compound 5 | 6.2 | 1,900 |
| Example 3 | Compound 9 | 6.4 | 1,800 |
| Example 4 | Compound 13 | 6.2 | 1,900 |
| Comparative Example 1 | Comparative Compound | 7.3 | 1,400 |
| | C1 | | |
| Comparative Example 2 | Comparative Compound | 7.5 | 1,000 |
| | C2 | | |
| Comparative Example 3 | Comparative Compound | 7.6 | 1,300 |
| | C3 | | |

Referring to the results in Table 1, the organic electroluminescent devices of Examples 1 to 4 exhibited longer lifetimes than that of Comparative Example 1, in which the material included in the hole transport layer has a carbazole part (e.g., carbazole functional group) that is meta- to a phenylene group. In addition, the organic electroluminescent devices of Examples 1 to 4 exhibited longer lifetimes than that of Comparative Example 2, in which the material included in the hole transport layer does not include an amine part (e.g., amine moiety). In addition, the organic electroluminescent devices of Examples 1 to 4 exhibited longer lifetimes than that of Comparative Example 3, in which the material included in the hole transport layer has an electron-accepting pyridylene group as the connecting group to the amine moiety, and a carbazole part (e.g., carbazole functional group) that is bonded to the phenylene group para with respect to the amine moiety through the pyridylene group. In addition, a long lifetime was realized in the organic electroluminescent devices of Examples 2 and 4, in which a nitrogen atom at the 9th position of the azacarbazole moiety was bonded (e.g., coupled) to a meta position of the phenylene ring, with respect to the amine moiety.

Based on the results shown in Table 1 and without being bound by any particular theory, it is believed that a longer lifetime of an organic electroluminescent device can be realized when using the material for an organic electroluminescent device of embodiments of the present disclosure as the hole transport material, than when using compounds of the Comparative Examples 1 to 3. In the material for an organic electroluminescent device according to one of more embodiments of the present disclosure, an azacarbazole part (e.g., azacarbazole moiety) is introduced (e.g., coupled) to the meta position of a phenylene group which is bonded to an amine moiety, in order to take advantage of the long emission lifetime property of the amine while further improving the amorphousness and lifetime of the material. In addition, by introducing an azacarbazole group with high electron-accepting characteristics, the electron resistance of the material for an organic electroluminescent device may be increased, and the durability of the material may be improved. Consequently, the lifetime of the organic electroluminescent device may be further increased.

In the material for an organic electroluminescent device according to embodiments of the present disclosure, the degree of amorphousness of the material may be improved, and the electron resistance thereof may be improved by introducing (e.g., coupling) an azacarbazole part (e.g., azacarbazole moiety) to a meta position of a phenylene group with respect to an amine moiety. Thus, the carrier mobility may be increased, and a long lifetime of the organic electroluminescent device may be realized. Moreover, since the material for an organic electroluminescent device according to embodiments of the present disclosure has a relatively wide energy gap, application of the material in a red emission region may also be possible.

One or more embodiments of the present disclosure are directed toward a material for use in an electroluminescent device in which a long lifetime is realized, along with an organic electroluminescent device using the same. Embodiments of the present disclosure relate to the realization of the material for a long-lifetime organic electroluminescent device in the green to blue emission regions, where the material is included in at least one lamination layer positioned between an emission layer and an anode. In some embodiments, the material for an organic electroluminescent device may realize a long lifetime by including an azacarbazole part (e.g., azacarbazole moiety) coupled to a meta position of a phenylene group which is bonded, directly or through a connecting group (e.g., L₁ in Formula 1), to the nitrogen atom (N) of an amine moiety, thereby improving the degree of amorphousness of the material and resulting in increased carrier mobility and improved electron resistance to enhance the durability of the material for use in an electroluminescent device.

As used herein, expressions such as “at least one of,” “one of,” “at least one selected from,” and “one selected from,” when preceding a list of elements, modify the entire list of elements and do not modify the individual elements of the list. Further, the use of “may” when describing embodiments of the present invention refers to “one or more embodiments of the present invention.”

In addition, as used herein, the terms “use,” “using,” and “used” may be considered synonymous with the terms “utilize,” “utilizing,” and “utilized,” respectively.

As used herein, the terms “substantially,” “about,” and similar terms are used as terms of approximation and not as terms of degree, and are intended to account for the inherent deviations in measured or calculated values that would be recognized by those of ordinary skill in the art.

Also, any numerical range recited herein is intended to include all subranges of the same numerical precision subsumed within the recited range. For example, a range of “1.0 to 10.0” is intended to include all subranges between (and including) the recited minimum value of 1.0 and the recited maximum value of 10.0, that is, having a minimum value equal to or greater than 1.0 and a maximum value equal to or less than 10.0, such as, for example, 2.4 to 7.6. Any maximum numerical limitation recited herein is intended to include all lower numerical limitations subsumed therein and any minimum numerical limitation recited in this specification is intended to include all higher numerical limitations subsumed therein. Accordingly, Applicant reserves the right to amend this specification, including the claims, to expressly recite any sub-range subsumed within the ranges expressly recited herein. All such ranges are intended to be inherently described in this specification such that amending to expressly recite any such subranges would comply with the requirements of 35 U.S.C. §112(a) and 35 U.S.C. §132(a).

The above-disclosed subject matter is to be considered illustrative and not restrictive, and the appended claims and equivalents thereof are intended to cover all such modifications, enhancements, and other embodiments, which fall within the true spirit and scope of the present disclosure. Thus, to the maximum extent allowed by law, the scope of the present disclosure is to be determined by the broadest permissible interpretation of the following claims and their equivalents, and shall not be restricted or limited by the foregoing detailed description.

What is claimed is:

1. A material for an organic electroluminescent device represented by the following Formula 1: 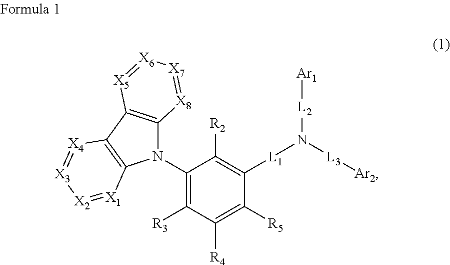
wherein:
X₁ to X₈ are each independently selected from N and CR₁, and at least one of X₁ to X₈ is N;
R₁ to R₅ are each independently selected from a substituted or unsubstituted aryl group having 6 to 30 carbon atoms for forming a ring, a substituted or unsubstituted heteroaryl group having 1 to 30 carbon atoms for forming a ring, an alkyl group having 1 to 15 carbon atoms, a substituted or unsubstituted silyl group, a halogen atom, hydrogen, and deuterium;
L₁ to L₃ are each independently a single bond or a divalent group selected from a substituted or unsubstituted alkylene group, a substituted or unsubstituted aralkylene group, a substituted or unsubstituted arylene group, a substituted or unsubstituted heteroarylene group, and a silyl group; and
Ar₁ and Ar₂ are each independently selected from a substituted or unsubstituted aryl group having 6 to 30 carbon atoms for forming a ring and a substituted or unsubstituted heteroaryl group having 1 to 30 carbon atoms for forming a ring.

2. A material for an organic electroluminescent device represented by the following Formula 2: 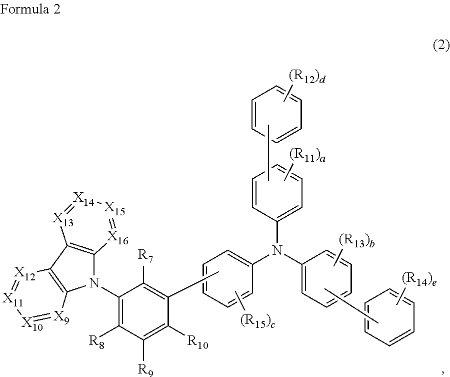
wherein:
X₉ to X₁₆ are each independently selected from N and CR₆, and at least one of X₉ to X₁₆ is N;
R₆ to R₁₅ are each independently selected from a substituted or unsubstituted aryl group having 6 to 30 carbon atoms for forming a ring, a substituted or unsubstituted heteroaryl group having 1 to 30 carbon atoms for forming a ring, an alkyl group having 1 to 15 carbon atoms, a substituted or unsubstituted silyl group, a halogen atom, hydrogen, and deuterium; and
a to c are each independently an integer selected from 0 to 4, and d and e are each independently an integer selected from 0 to 5.

3. The material of claim 2, wherein the material comprises at least one selected from Compounds 1 to 60:

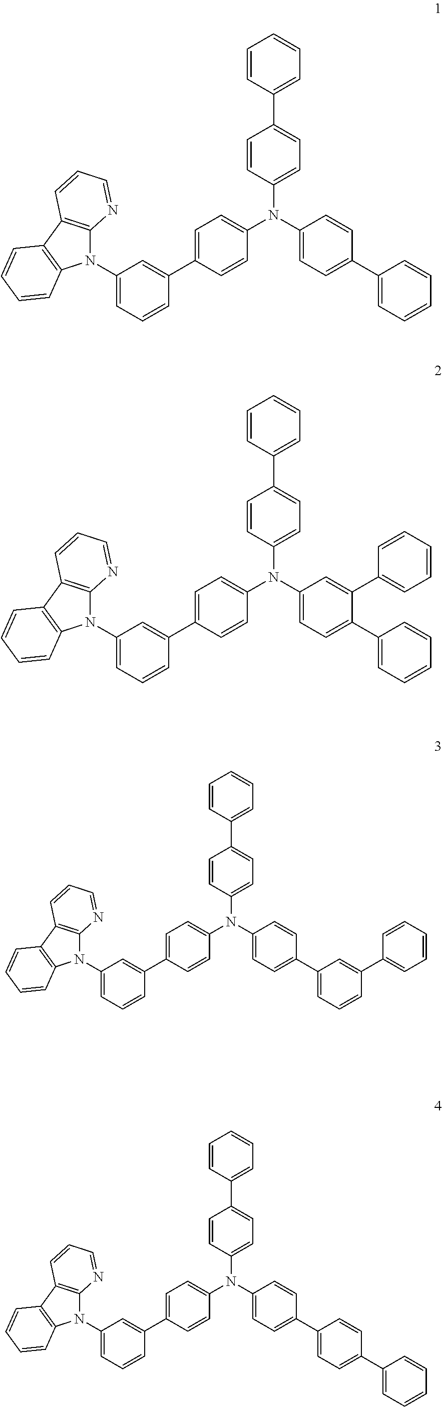 [SMILES: c1ccc(-c2ccc(N(c3ccc(-c4ccccc4)cc3)c3ccc(-c4cccc(-n5c6ccccc6c6cccnc65)c4)cc3)cc2)cc1 ; c1ccc(-c2ccc(N(c3ccc(-c4cccc(-n5c6ccccc6c6cccnc65)c4)cc3)c3ccc(-c4ccccc4)c(-c4ccccc4)c3)cc2)cc1 ; c1ccc(-c2ccc(N(c3ccc(-c4cccc(-c5ccccc5)c4)cc3)c3ccc(-c4cccc(-n5c6ccccc6c6cccnc65)c4)cc3)cc2)cc1 ; c1ccc(-c2ccc(-c3ccc(N(c4ccc(-c5ccccc5)cc4)c4ccc(-c5cccc(-n6c7ccccc7c7cccnc76)c5)cc4)cc3)cc2)cc1]

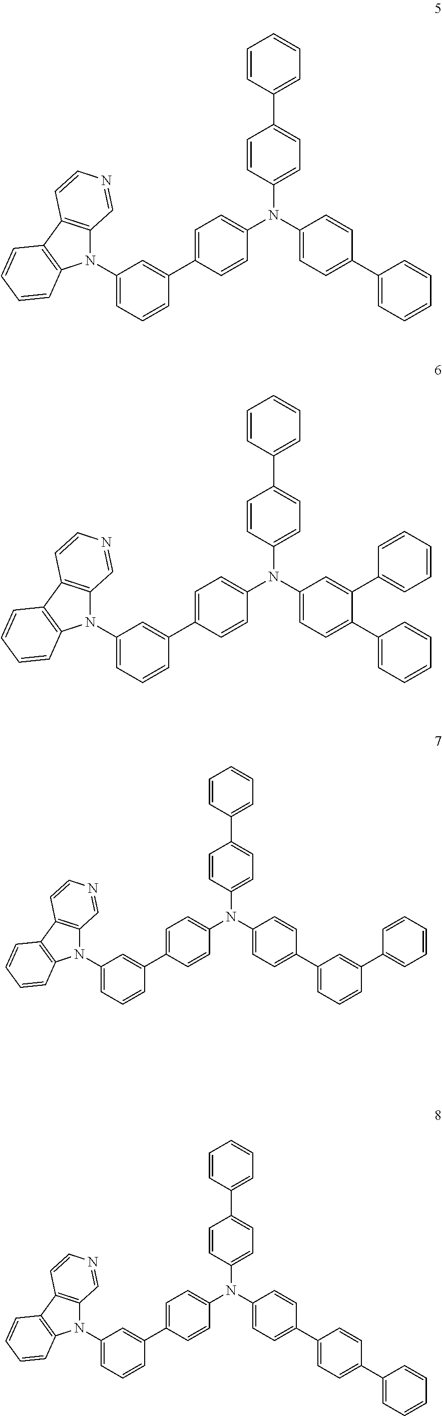 [SMILES: c1ccc(-c2ccc(N(c3ccc(-c4ccccc4)cc3)c3ccc(-c4cccc(-n5c6ccccc6c6ccncc65)c4)cc3)cc2)cc1 ; c1ccc(-c2ccc(N(c3ccc(-c4cccc(-n5c6ccccc6c6ccncc65)c4)cc3)c3ccc(-c4ccccc4)c(-c4ccccc4)c3)cc2)cc1 ; c1ccc(-c2ccc(N(c3ccc(-c4cccc(-c5ccccc5)c4)cc3)c3ccc(-c4cccc(-n5c6ccccc6c6ccncc65)c4)cc3)cc2)cc1 ; c1ccc(-c2ccc(-c3ccc(N(c4ccc(-c5ccccc5)cc4)c4ccc(-c5cccc(-n6c7ccccc7c7ccncc76)c5)cc4)cc3)cc2)cc1]

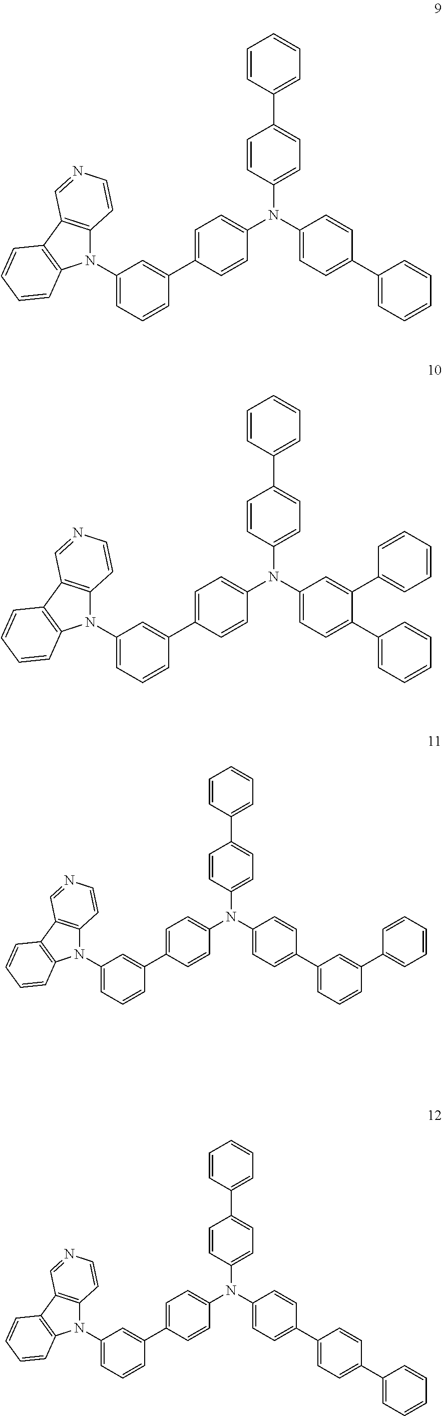 [SMILES: c1ccc(-c2ccc(N(c3ccc(-c4ccccc4)cc3)c3ccc(-c4cccc(-n5c6ccccc6c6cnccc65)c4)cc3)cc2)cc1 ; c1ccc(-c2ccc(N(c3ccc(-c4cccc(-n5c6ccccc6c6cnccc65)c4)cc3)c3ccc(-c4ccccc4)c(-c4ccccc4)c3)cc2)cc1 ; c1ccc(-c2ccc(N(c3ccc(-c4cccc(-c5ccccc5)c4)cc3)c3ccc(-c4cccc(-n5c6ccccc6c6cnccc65)c4)cc3)cc2)cc1 ; c1ccc(-c2ccc(-c3ccc(N(c4ccc(-c5ccccc5)cc4)c4ccc(-c5cccc(-n6c7ccccc7c7cnccc76)c5)cc4)cc3)cc2)cc1]

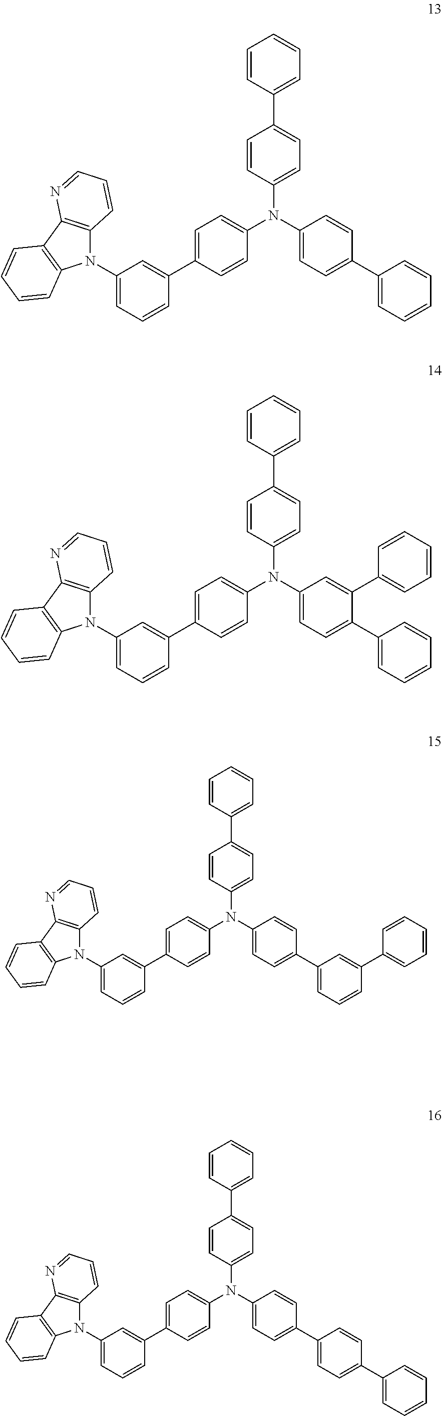 [SMILES: c1ccc(-c2ccc(N(c3ccc(-c4ccccc4)cc3)c3ccc(-c4cccc(-n5c6ccccc6c6ncccc65)c4)cc3)cc2)cc1 ; c1ccc(-c2ccc(N(c3ccc(-c4cccc(-n5c6ccccc6c6ncccc65)c4)cc3)c3ccc(-c4ccccc4)c(-c4ccccc4)c3)cc2)cc1 ; c1ccc(-c2ccc(N(c3ccc(-c4cccc(-c5ccccc5)c4)cc3)c3ccc(-c4cccc(-n5c6ccccc6c6ncccc65)c4)cc3)cc2)cc1 ; c1ccc(-c2ccc(-c3ccc(N(c4ccc(-c5ccccc5)cc4)c4ccc(-c5cccc(-n6c7ccccc7c7ncccc76)c5)cc4)cc3)cc2)cc1]

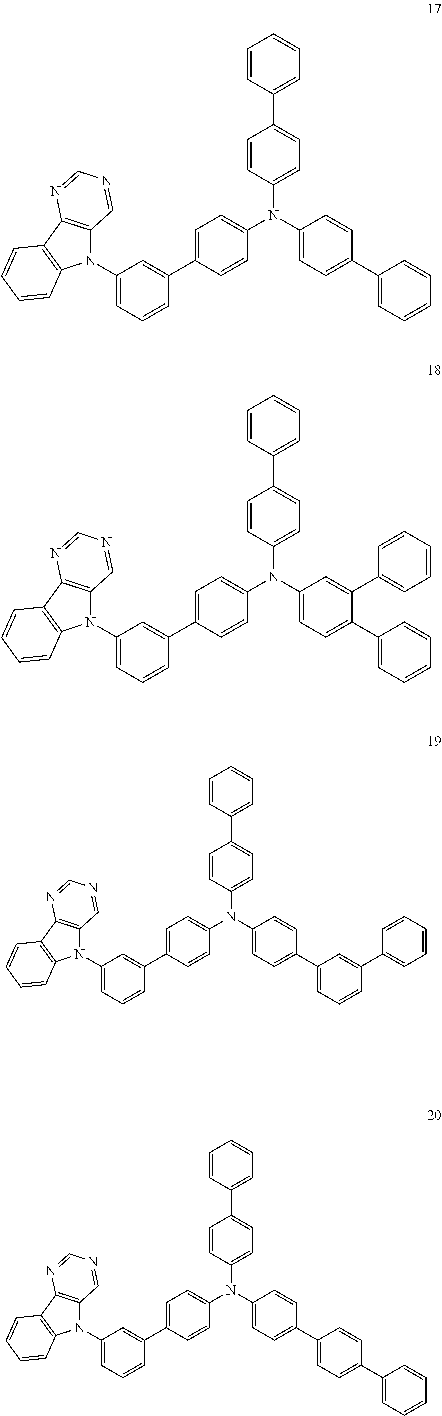 [SMILES: c1ccc(-c2ccc(N(c3ccc(-c4ccccc4)cc3)c3ccc(-c4cccc(-n5c6ccccc6c6ncncc65)c4)cc3)cc2)cc1 ; c1ccc(-c2ccc(N(c3ccc(-c4cccc(-n5c6ccccc6c6ncncc65)c4)cc3)c3ccc(-c4ccccc4)c(-c4ccccc4)c3)cc2)cc1 ; c1ccc(-c2ccc(N(c3ccc(-c4cccc(-c5ccccc5)c4)cc3)c3ccc(-c4cccc(-n5c6ccccc6c6ncncc65)c4)cc3)cc2)cc1 ; c1ccc(-c2ccc(-c3ccc(N(c4ccc(-c5ccccc5)cc4)c4ccc(-c5cccc(-n6c7ccccc7c7ncncc76)c5)cc4)cc3)cc2)cc1]

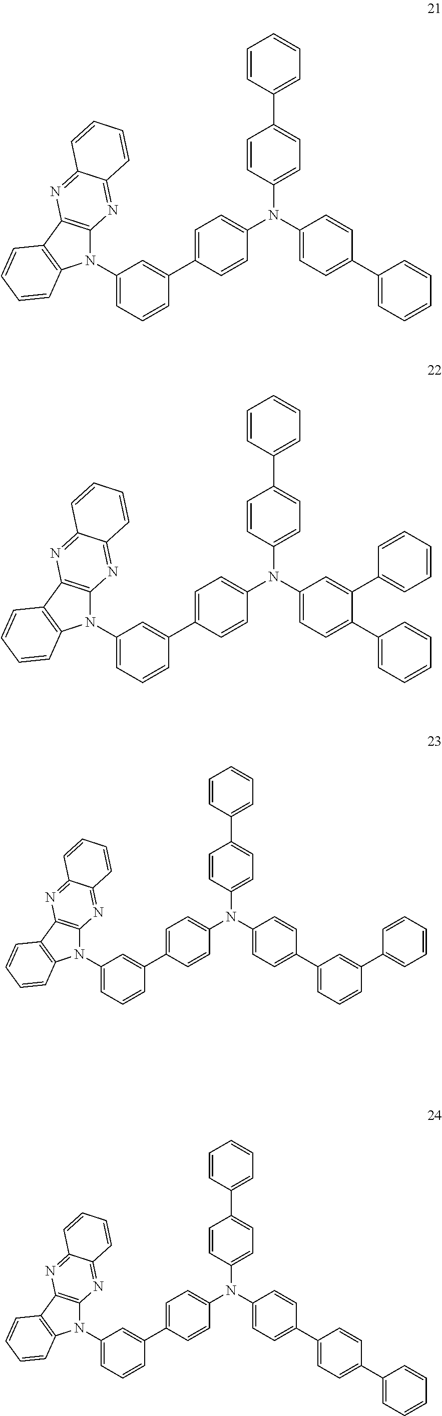 [SMILES: c1ccc(-c2ccc(N(c3ccc(-c4ccccc4)cc3)c3ccc(-c4cccc(-n5c6ccccc6c6nc7ccccc7nc65)c4)cc3)cc2)cc1 ; c1ccc(-c2ccc(N(c3ccc(-c4cccc(-n5c6ccccc6c6nc7ccccc7nc65)c4)cc3)c3ccc(-c4ccccc4)c(-c4ccccc4)c3)cc2)cc1 ; c1ccc(-c2ccc(N(c3ccc(-c4cccc(-c5ccccc5)c4)cc3)c3ccc(-c4cccc(-n5c6ccccc6c6nc7ccccc7nc65)c4)cc3)cc2)cc1 ; c1ccc(-c2ccc(-c3ccc(N(c4ccc(-c5ccccc5)cc4)c4ccc(-c5cccc(-n6c7ccccc7c7nc8ccccc8nc76)c5)cc4)cc3)cc2)cc1]

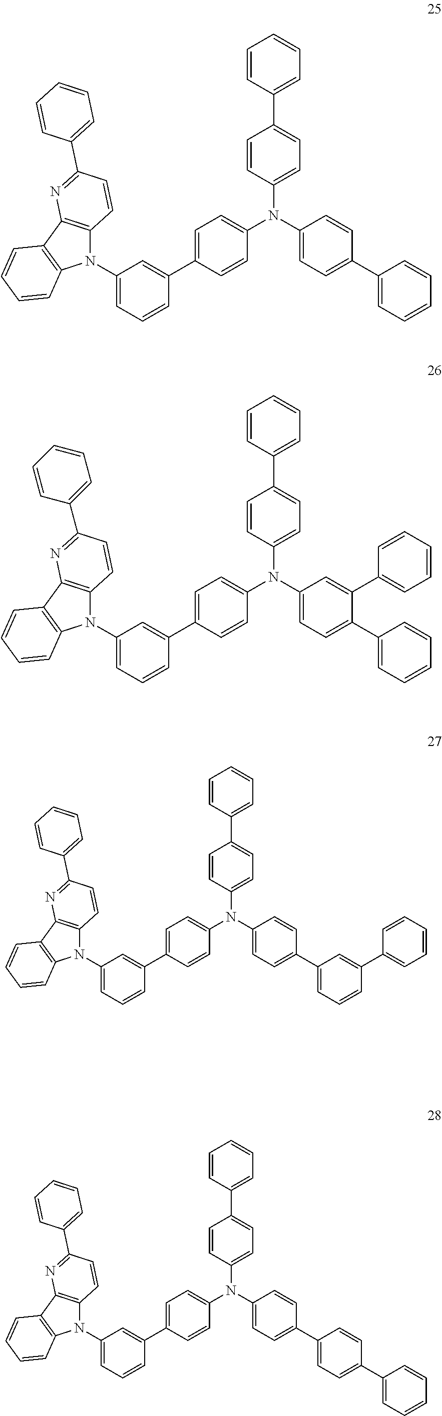 [SMILES: c1ccc(-c2ccc(N(c3ccc(-c4ccccc4)cc3)c3ccc(-c4cccc(-n5c6ccccc6c6nc(-c7ccccc7)ccc65)c4)cc3)cc2)cc1 ; c1ccc(-c2ccc(N(c3ccc(-c4cccc(-n5c6ccccc6c6nc(-c7ccccc7)ccc65)c4)cc3)c3ccc(-c4ccccc4)c(-c4ccccc4)c3)cc2)cc1 ; c1ccc(-c2ccc(N(c3ccc(-c4cccc(-c5ccccc5)c4)cc3)c3ccc(-c4cccc(-n5c6ccccc6c6nc(-c7ccccc7)ccc65)c4)cc3)cc2)cc1 ; c1ccc(-c2ccc(-c3ccc(N(c4ccc(-c5ccccc5)cc4)c4ccc(-c5cccc(-n6c7ccccc7c7nc(-c8ccccc8)ccc76)c5)cc4)cc3)cc2)cc1]

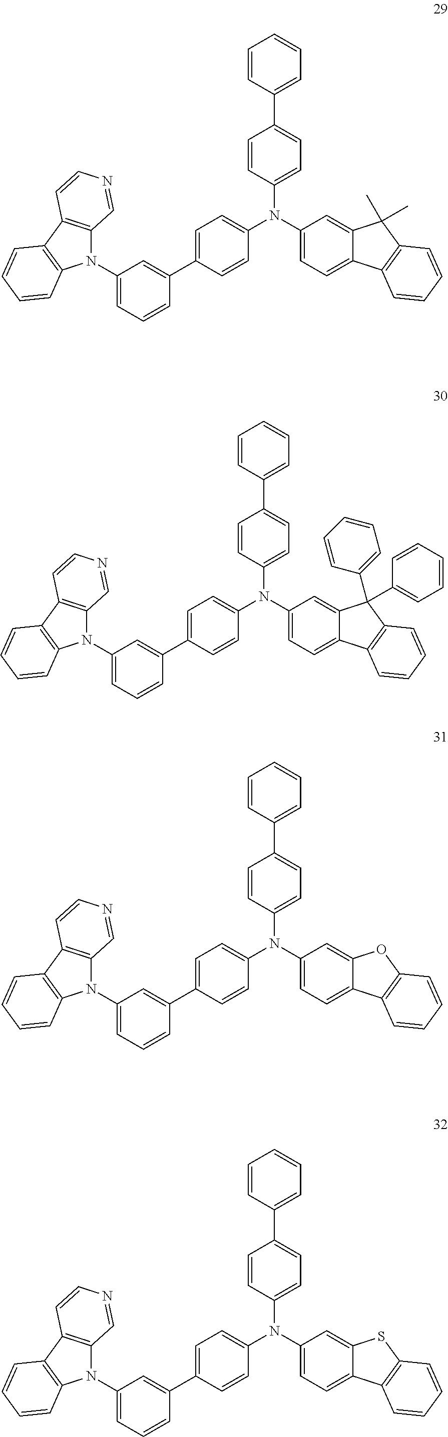 [SMILES: CC1(C)c2ccccc2-c2ccc(N(c3ccc(-c4ccccc4)cc3)c3ccc(-c4cccc(-n5c6ccccc6c6ccncc65)c4)cc3)cc21 ; c1ccc(-c2ccc(N(c3ccc(-c4cccc(-n5c6ccccc6c6ccncc65)c4)cc3)c3ccc4c(c3)C(c3ccccc3)(c3ccccc3)c3ccccc3-4)cc2)cc1 ; c1ccc(-c2ccc(N(c3ccc(-c4cccc(-n5c6ccccc6c6ccncc65)c4)cc3)c3ccc4c(c3)oc3ccccc34)cc2)cc1 ; c1ccc(-c2ccc(N(c3ccc(-c4cccc(-n5c6ccccc6c6ccncc65)c4)cc3)c3ccc4c(c3)sc3ccccc34)cc2)cc1]

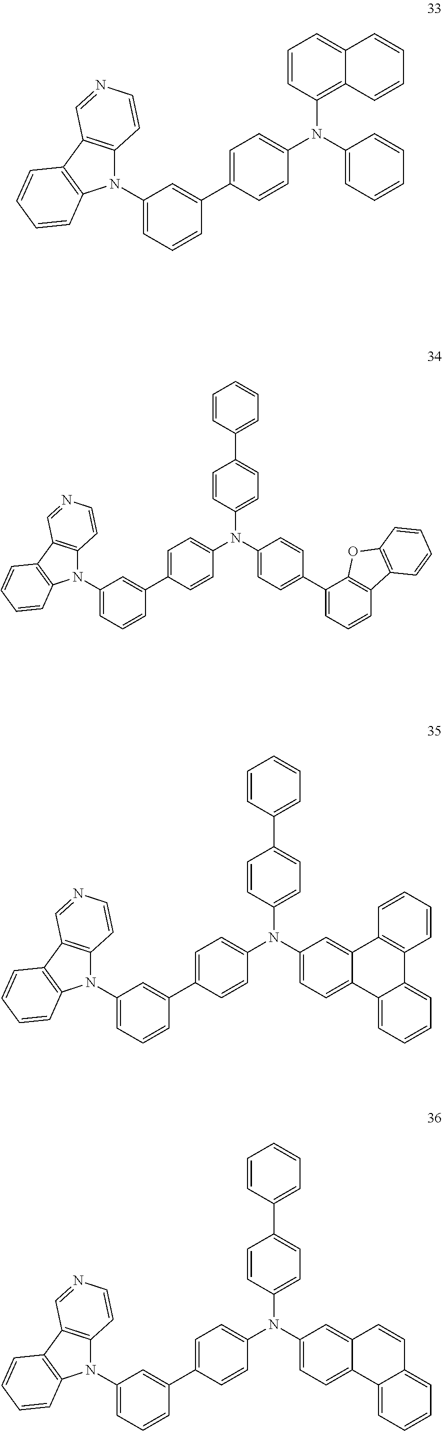 [SMILES: c1ccc(N(c2ccc(-c3cccc(-n4c5ccccc5c5cnccc54)c3)cc2)c2cccc3ccccc23)cc1 ; c1ccc(-c2ccc(N(c3ccc(-c4cccc(-n5c6ccccc6c6cnccc65)c4)cc3)c3ccc(-c4cccc5c4oc4ccccc45)cc3)cc2)cc1 ; c1ccc(-c2ccc(N(c3ccc(-c4cccc(-n5c6ccccc6c6cnccc65)c4)cc3)c3ccc4c5ccccc5c5ccccc5c4c3)cc2)cc1 ; c1ccc(-c2ccc(N(c3ccc(-c4cccc(-n5c6ccccc6c6cnccc65)c4)cc3)c3ccc4c(ccc5ccccc54)c3)cc2)cc1]

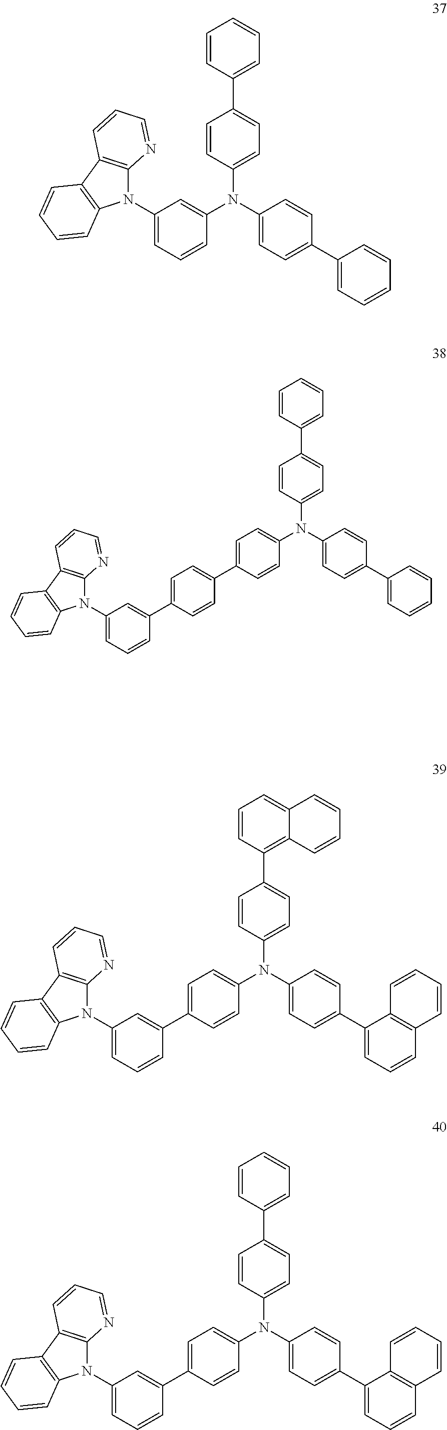 [SMILES: c1ccc(-c2ccc(N(c3ccc(-c4ccccc4)cc3)c3cccc(-n4c5ccccc5c5cccnc54)c3)cc2)cc1 ; c1ccc(-c2ccc(N(c3ccc(-c4ccccc4)cc3)c3ccc(-c4ccc(-c5cccc(-n6c7ccccc7c7cccnc76)c5)cc4)cc3)cc2)cc1 ; c1cc(-c2ccc(N(c3ccc(-c4cccc5ccccc45)cc3)c3ccc(-c4cccc5ccccc45)cc3)cc2)cc(-n2c3ccccc3c3cccnc32)c1 ; c1ccc(-c2ccc(N(c3ccc(-c4cccc(-n5c6ccccc6c6cccnc65)c4)cc3)c3ccc(-c4cccc5ccccc45)cc3)cc2)cc1]

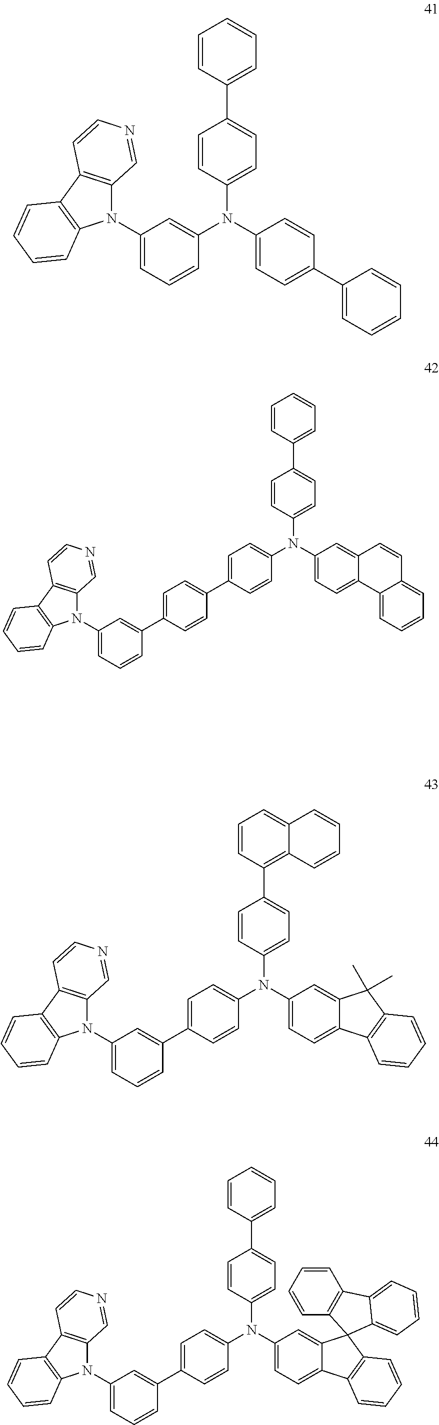 [SMILES: c1ccc(-c2ccc(N(c3ccc(-c4ccccc4)cc3)c3cccc(-n4c5ccccc5c5ccncc54)c3)cc2)cc1 ; c1ccc(-c2ccc(N(c3ccc(-c4ccc(-c5cccc(-n6c7ccccc7c7ccncc76)c5)cc4)cc3)c3ccc4c(ccc5ccccc54)c3)cc2)cc1 ; CC1(C)c2ccccc2-c2ccc(N(c3ccc(-c4cccc(-n5c6ccccc6c6ccncc65)c4)cc3)c3ccc(-c4cccc5ccccc45)cc3)cc21 ; c1ccc(-c2ccc(N(c3ccc(-c4cccc(-n5c6ccccc6c6ccncc65)c4)cc3)c3ccc4c(c3)C3(c5ccccc5-c5ccccc53)c3ccccc3-4)cc2)cc1]

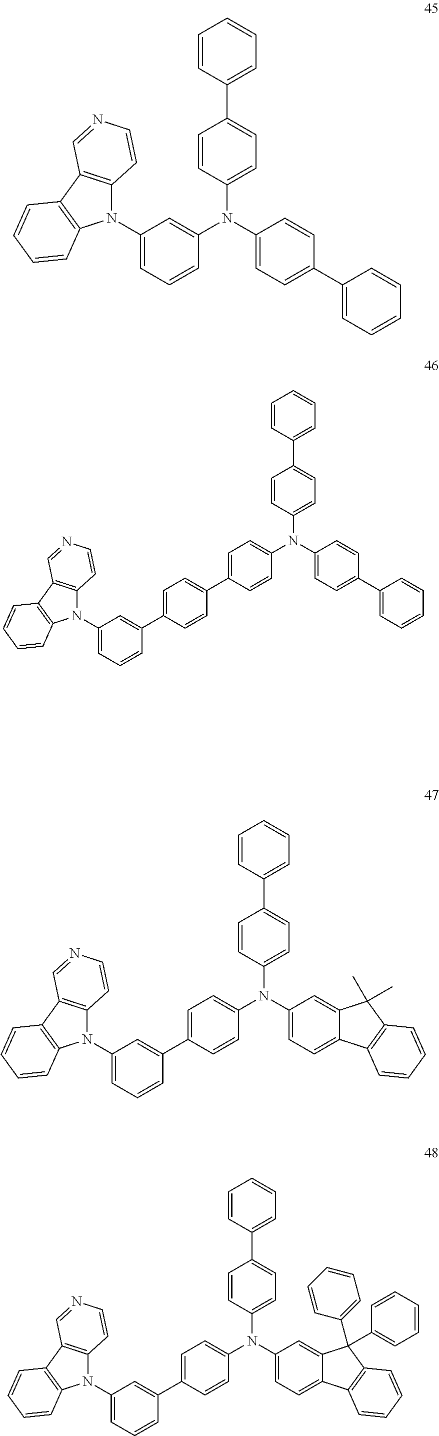 [SMILES: c1ccc(-c2ccc(N(c3ccc(-c4ccccc4)cc3)c3cccc(-n4c5ccccc5c5cnccc54)c3)cc2)cc1 ; c1ccc(-c2ccc(N(c3ccc(-c4ccccc4)cc3)c3ccc(-c4ccc(-c5cccc(-n6c7ccccc7c7cnccc76)c5)cc4)cc3)cc2)cc1 ; CC1(C)c2ccccc2-c2ccc(N(c3ccc(-c4ccccc4)cc3)c3ccc(-c4cccc(-n5c6ccccc6c6cnccc65)c4)cc3)cc21 ; c1ccc(-c2ccc(N(c3ccc(-c4cccc(-n5c6ccccc6c6cnccc65)c4)cc3)c3ccc4c(c3)C(c3ccccc3)(c3ccccc3)c3ccccc3-4)cc2)cc1]

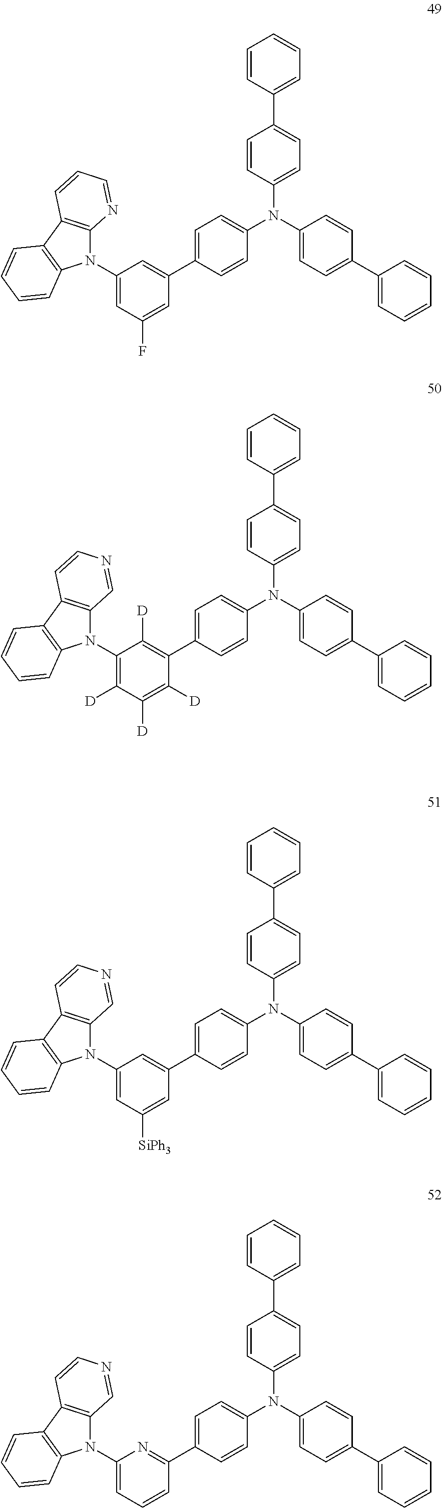

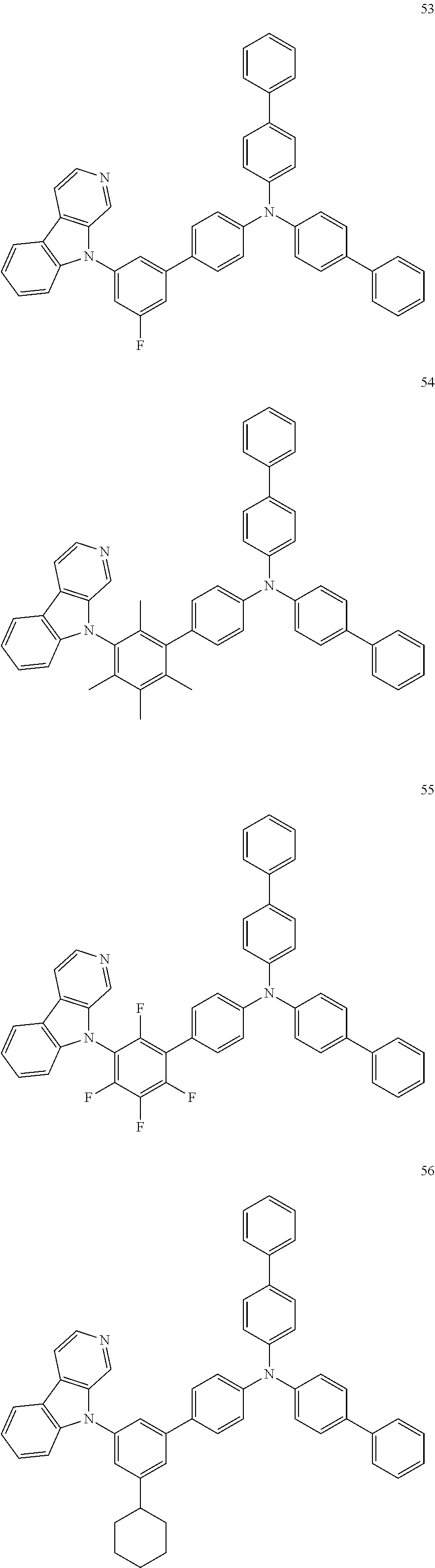 [SMILES: Fc1cc(-c2ccc(N(c3ccc(-c4ccccc4)cc3)c3ccc(-c4ccccc4)cc3)cc2)cc(-n2c3ccccc3c3ccncc32)c1 ; Cc1c(C)c(-c2ccc(N(c3ccc(-c4ccccc4)cc3)c3ccc(-c4ccccc4)cc3)cc2)c(C)c(-n2c3ccccc3c3ccncc32)c1C ; Fc1c(F)c(-c2ccc(N(c3ccc(-c4ccccc4)cc3)c3ccc(-c4ccccc4)cc3)cc2)c(F)c(-n2c3ccccc3c3ccncc32)c1F ; c1ccc(-c2ccc(N(c3ccc(-c4ccccc4)cc3)c3ccc(-c4cc(C5CCCCC5)cc(-n5c6ccccc6c6ccncc65)c4)cc3)cc2)cc1]

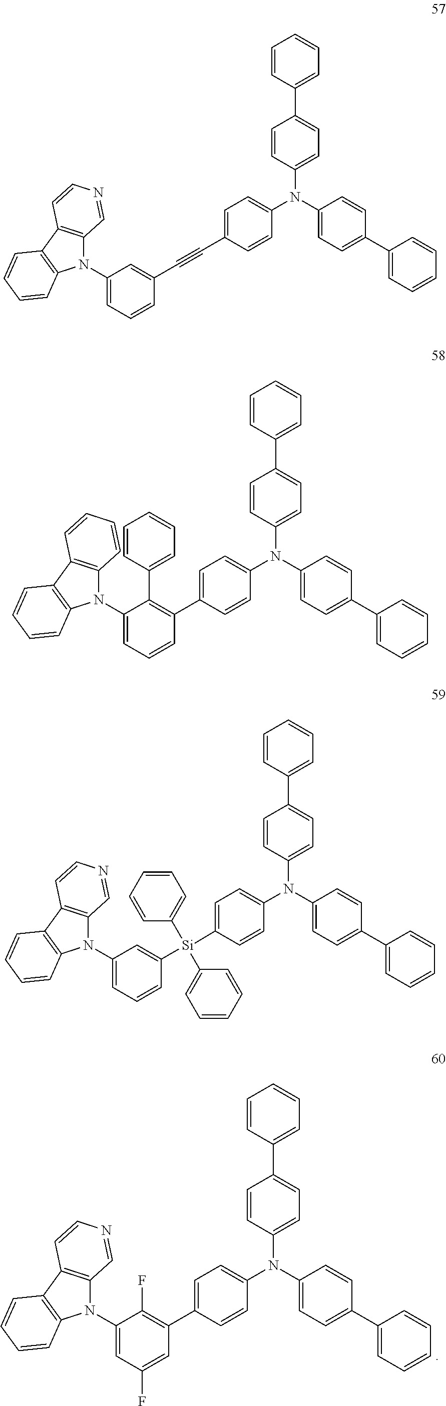 [SMILES: C(#Cc1cccc(-n2c3ccccc3c3ccncc32)c1)c1ccc(N(c2ccc(-c3ccccc3)cc2)c2ccc(-c3ccccc3)cc2)cc1 ; c1ccc(-c2ccc(N(c3ccc(-c4ccccc4)cc3)c3ccc(-c4cccc(-n5c6ccccc6c6ccccc65)c4-c4ccccc4)cc3)cc2)cc1 ; c1ccc(-c2ccc(N(c3ccc(-c4ccccc4)cc3)c3ccc([Si](c4ccccc4)(c4ccccc4)c4cccc(-n5c6ccccc6c6ccncc65)c4)cc3)cc2)cc1 ; Fc1cc(-c2ccc(N(c3ccc(-c4ccccc4)cc3)c3ccc(-c4ccccc4)cc3)cc2)c(F)c(-n2c3ccccc3c3ccncc32)c1]

4. An organic electroluminescent device comprising:
an anode,
a cathode,
an emission layer between the anode and the cathode, and
a plurality of lamination layers between the emission layer and the anode,
wherein at least one layer selected from the plurality of lamination layers comprises a material for an organic electroluminescent device represented by the following Formula 1: 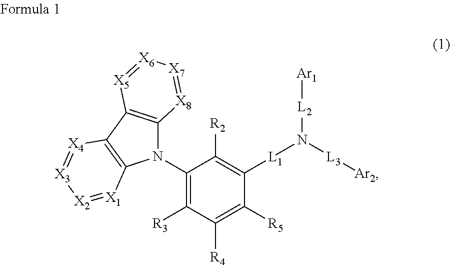
wherein:
X₁ to X₈ are each independently selected from N and CR₁, and at least one of X₁ to X₈ is N;
R₁ to R₅ are each independently selected from a substituted or unsubstituted aryl group having 6 to 30 carbon atoms for forming a ring, a substituted or unsubstituted heteroaryl group having 1 to 30 carbon atoms for forming a ring, an alkyl group having 1 to 15 carbon atoms, a substituted or unsubstituted silyl group, a halogen atom, hydrogen, and deuterium;
L₁ to L₃ are each independently a single bond or a divalent group selected from a substituted or unsubstituted alkylene group, a substituted or unsubstituted aralkylene group, a substituted or unsubstituted arylene group, a substituted or unsubstituted heteroarylene group, and a silyl group; and
Ar₁ and Ar₂ are each independently selected from a substituted or unsubstituted aryl group having 6 to 30 carbon atoms for forming a ring and a substituted or unsubstituted heteroaryl group having 1 to 30 carbon atoms for forming a ring.

5. The organic electroluminescent device of claim 4, wherein the material is represented by Formula 2: 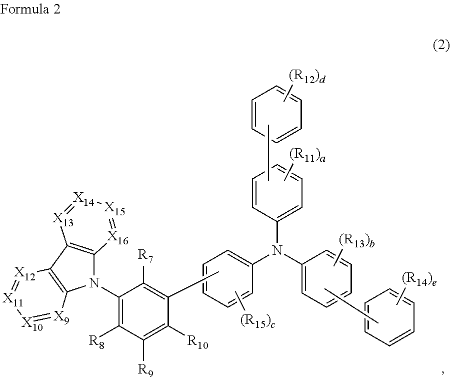
wherein:
X₉ to X₁₆ are each independently selected from N and CR₆, and at least one of X₉ to X₁₆ is N;
R₆ to R₁₅ are each independently selected from a substituted or unsubstituted aryl group having 6 to 30 carbon atoms for forming a ring, a substituted or unsubstituted heteroaryl group having 1 to 30 carbon atoms for forming a ring, an alkyl group having 1 to 15 carbon atoms, a substituted or unsubstituted silyl group, a halogen atom, hydrogen, and deuterium; and
a to c are each independently an integer selected from 0 to 4, and d and e are each independently an integer selected from 0 to 5.

6. The organic electroluminescent device of claim 4, wherein the material comprises at least one selected from Compounds 1 to 60: 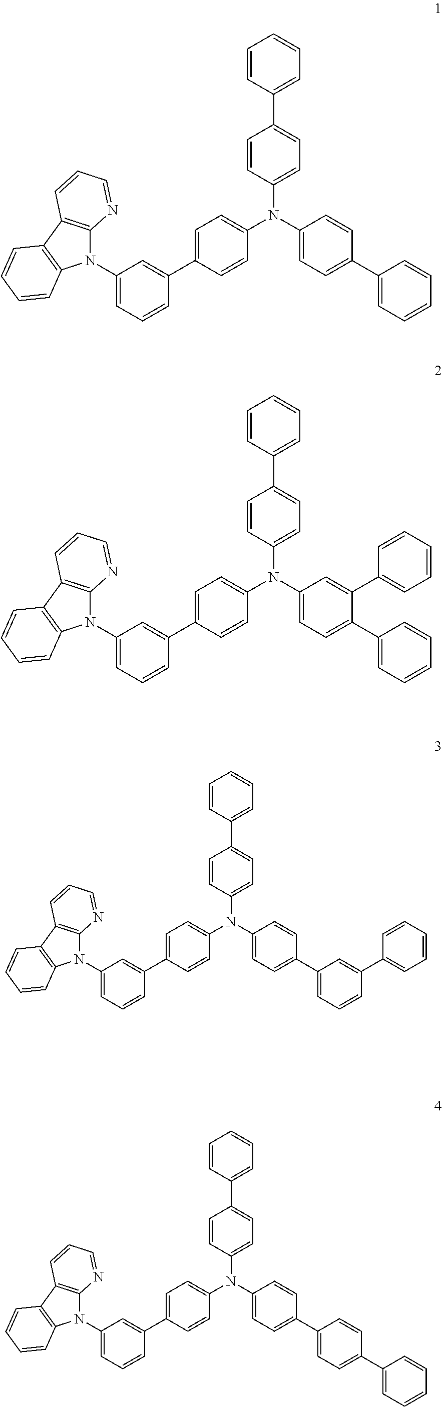 [SMILES: c1ccc(-c2ccc(N(c3ccc(-c4ccccc4)cc3)c3ccc(-c4cccc(-n5c6ccccc6c6cccnc65)c4)cc3)cc2)cc1 ; c1ccc(-c2ccc(N(c3ccc(-c4cccc(-n5c6ccccc6c6cccnc65)c4)cc3)c3ccc(-c4ccccc4)c(-c4ccccc4)c3)cc2)cc1 ; c1ccc(-c2ccc(N(c3ccc(-c4cccc(-c5ccccc5)c4)cc3)c3ccc(-c4cccc(-n5c6ccccc6c6cccnc65)c4)cc3)cc2)cc1 ; c1ccc(-c2ccc(-c3ccc(N(c4ccc(-c5ccccc5)cc4)c4ccc(-c5cccc(-n6c7ccccc7c7cccnc76)c5)cc4)cc3)cc2)cc1] 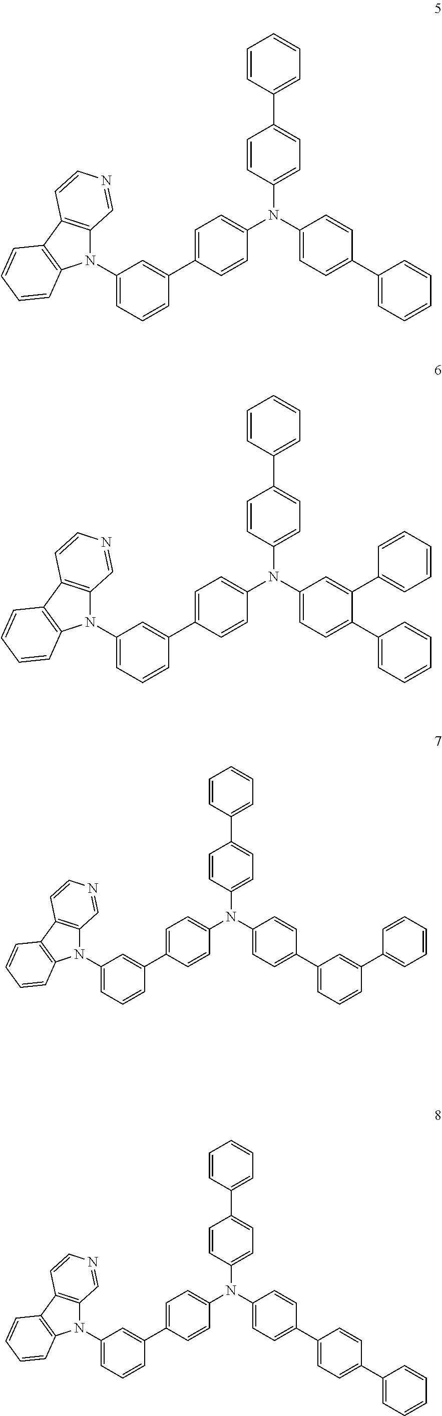 [SMILES: c1ccc(-c2ccc(N(c3ccc(-c4ccccc4)cc3)c3ccc(-c4cccc(-n5c6ccccc6c6ccncc65)c4)cc3)cc2)cc1 ; c1ccc(-c2ccc(N(c3ccc(-c4cccc(-n5c6ccccc6c6ccncc65)c4)cc3)c3ccc(-c4ccccc4)c(-c4ccccc4)c3)cc2)cc1 ; c1ccc(-c2ccc(N(c3ccc(-c4cccc(-c5ccccc5)c4)cc3)c3ccc(-c4cccc(-n5c6ccccc6c6ccncc65)c4)cc3)cc2)cc1 ; c1ccc(-c2ccc(-c3ccc(N(c4ccc(-c5ccccc5)cc4)c4ccc(-c5cccc(-n6c7ccccc7c7ccncc76)c5)cc4)cc3)cc2)cc1] 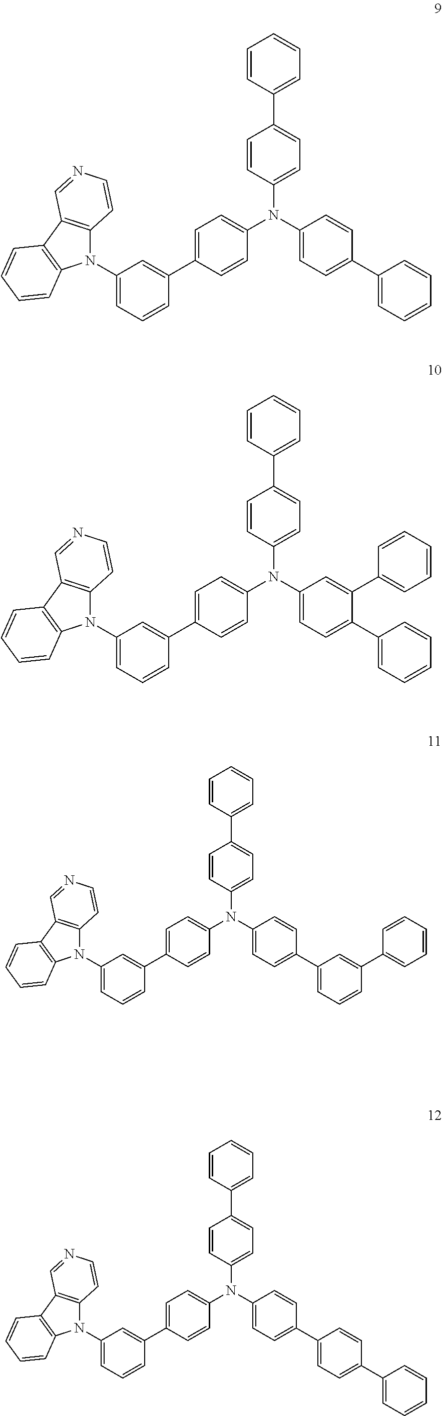 [SMILES: c1ccc(-c2ccc(N(c3ccc(-c4ccccc4)cc3)c3ccc(-c4cccc(-n5c6ccccc6c6cnccc65)c4)cc3)cc2)cc1 ; c1ccc(-c2ccc(N(c3ccc(-c4cccc(-n5c6ccccc6c6cnccc65)c4)cc3)c3ccc(-c4ccccc4)c(-c4ccccc4)c3)cc2)cc1 ; c1ccc(-c2ccc(N(c3ccc(-c4cccc(-c5ccccc5)c4)cc3)c3ccc(-c4cccc(-n5c6ccccc6c6cnccc65)c4)cc3)cc2)cc1 ; c1ccc(-c2ccc(-c3ccc(N(c4ccc(-c5ccccc5)cc4)c4ccc(-c5cccc(-n6c7ccccc7c7cnccc76)c5)cc4)cc3)cc2)cc1] 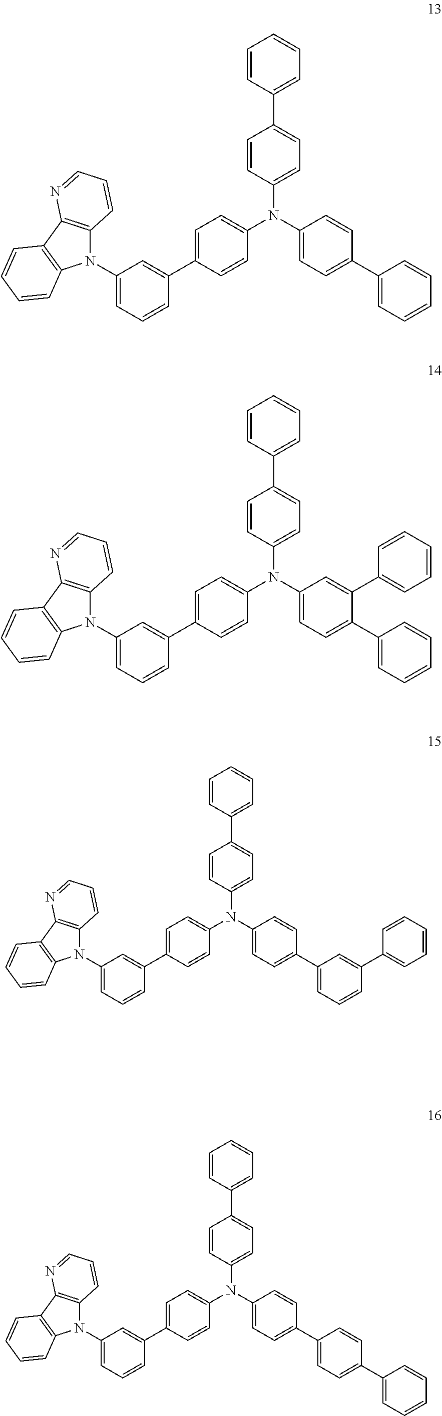 [SMILES: c1ccc(-c2ccc(N(c3ccc(-c4ccccc4)cc3)c3ccc(-c4cccc(-n5c6ccccc6c6ncccc65)c4)cc3)cc2)cc1 ; c1ccc(-c2ccc(N(c3ccc(-c4cccc(-n5c6ccccc6c6ncccc65)c4)cc3)c3ccc(-c4ccccc4)c(-c4ccccc4)c3)cc2)cc1 ; c1ccc(-c2ccc(N(c3ccc(-c4cccc(-c5ccccc5)c4)cc3)c3ccc(-c4cccc(-n5c6ccccc6c6ncccc65)c4)cc3)cc2)cc1 ; c1ccc(-c2ccc(-c3ccc(N(c4ccc(-c5ccccc5)cc4)c4ccc(-c5cccc(-n6c7ccccc7c7ncccc76)c5)cc4)cc3)cc2)cc1] 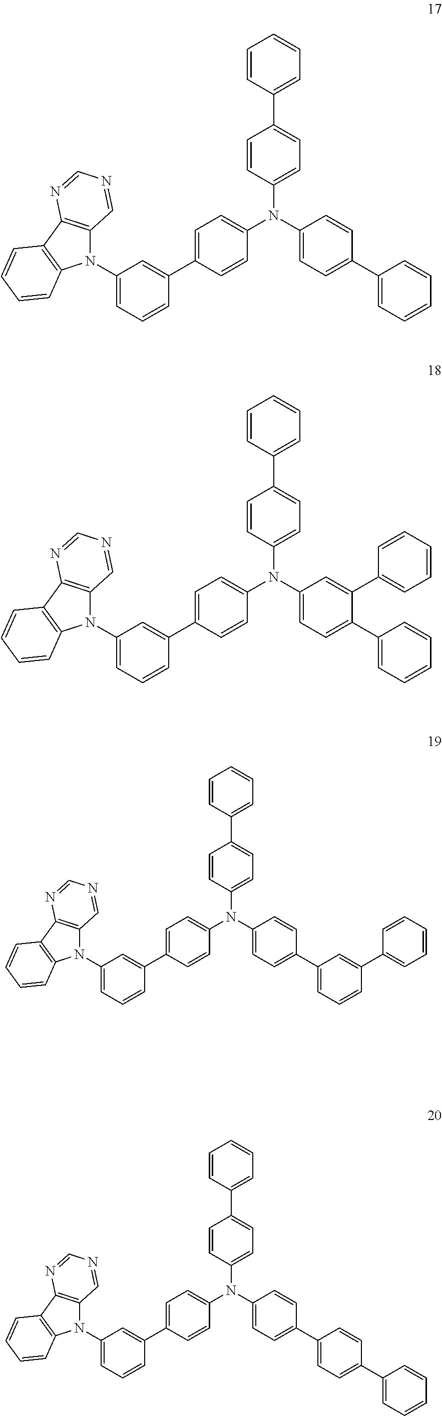 [SMILES: c1ccc(-c2ccc(N(c3ccc(-c4ccccc4)cc3)c3ccc(-c4cccc(-n5c6ccccc6c6ncncc65)c4)cc3)cc2)cc1 ; c1ccc(-c2ccc(N(c3ccc(-c4cccc(-n5c6ccccc6c6ncncc65)c4)cc3)c3ccc(-c4ccccc4)c(-c4ccccc4)c3)cc2)cc1 ; c1ccc(-c2ccc(N(c3ccc(-c4cccc(-c5ccccc5)c4)cc3)c3ccc(-c4cccc(-n5c6ccccc6c6ncncc65)c4)cc3)cc2)cc1 ; c1ccc(-c2ccc(-c3ccc(N(c4ccc(-c5ccccc5)cc4)c4ccc(-c5cccc(-n6c7ccccc7c7ncncc76)c5)cc4)cc3)cc2)cc1] 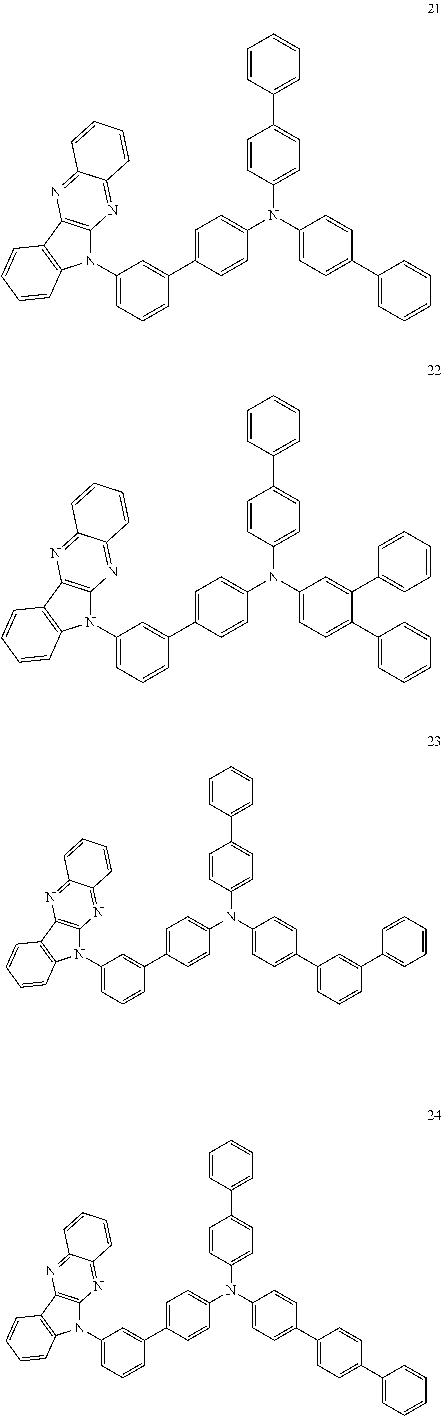 [SMILES: c1ccc(-c2ccc(N(c3ccc(-c4ccccc4)cc3)c3ccc(-c4cccc(-n5c6ccccc6c6nc7ccccc7nc65)c4)cc3)cc2)cc1 ; c1ccc(-c2ccc(N(c3ccc(-c4cccc(-n5c6ccccc6c6nc7ccccc7nc65)c4)cc3)c3ccc(-c4ccccc4)c(-c4ccccc4)c3)cc2)cc1 ; c1ccc(-c2ccc(N(c3ccc(-c4cccc(-c5ccccc5)c4)cc3)c3ccc(-c4cccc(-n5c6ccccc6c6nc7ccccc7nc65)c4)cc3)cc2)cc1 ; c1ccc(-c2ccc(-c3ccc(N(c4ccc(-c5ccccc5)cc4)c4ccc(-c5cccc(-n6c7ccccc7c7nc8ccccc8nc76)c5)cc4)cc3)cc2)cc1] 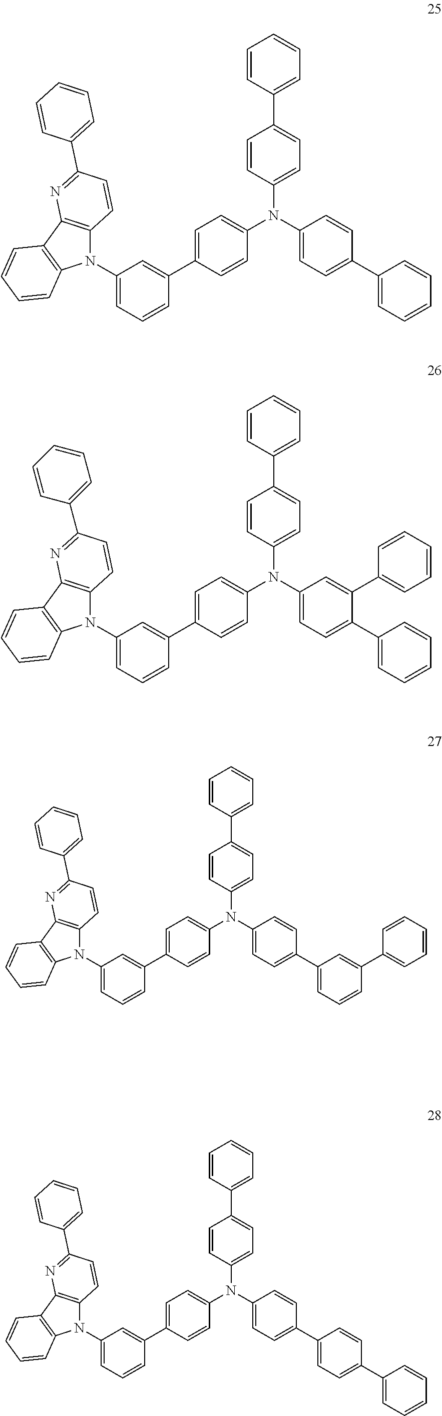 [SMILES: c1ccc(-c2ccc(N(c3ccc(-c4ccccc4)cc3)c3ccc(-c4cccc(-n5c6ccccc6c6nc(-c7ccccc7)ccc65)c4)cc3)cc2)cc1 ; c1ccc(-c2ccc(N(c3ccc(-c4cccc(-n5c6ccccc6c6nc(-c7ccccc7)ccc65)c4)cc3)c3ccc(-c4ccccc4)c(-c4ccccc4)c3)cc2)cc1 ; c1ccc(-c2ccc(N(c3ccc(-c4cccc(-c5ccccc5)c4)cc3)c3ccc(-c4cccc(-n5c6ccccc6c6nc(-c7ccccc7)ccc65)c4)cc3)cc2)cc1 ; c1ccc(-c2ccc(-c3ccc(N(c4ccc(-c5ccccc5)cc4)c4ccc(-c5cccc(-n6c7ccccc7c7nc(-c8ccccc8)ccc76)c5)cc4)cc3)cc2)cc1] 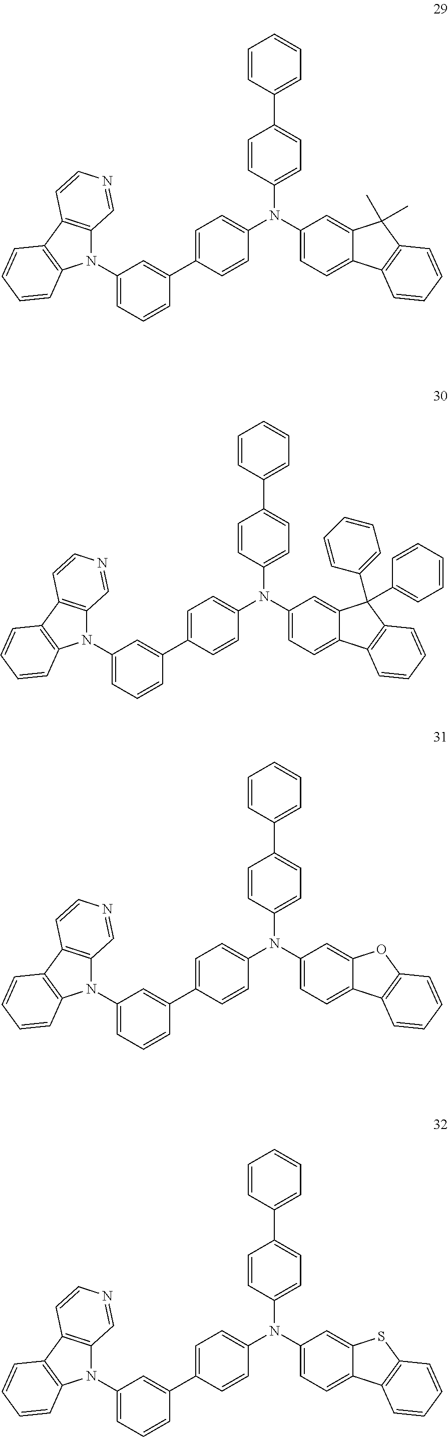 [SMILES: CC1(C)c2ccccc2-c2ccc(N(c3ccc(-c4ccccc4)cc3)c3ccc(-c4cccc(-n5c6ccccc6c6ccncc65)c4)cc3)cc21 ; c1ccc(-c2ccc(N(c3ccc(-c4cccc(-n5c6ccccc6c6ccncc65)c4)cc3)c3ccc4c(c3)C(c3ccccc3)(c3ccccc3)c3ccccc3-4)cc2)cc1 ; c1ccc(-c2ccc(N(c3ccc(-c4cccc(-n5c6ccccc6c6ccncc65)c4)cc3)c3ccc4c(c3)oc3ccccc34)cc2)cc1 ; c1ccc(-c2ccc(N(c3ccc(-c4cccc(-n5c6ccccc6c6ccncc65)c4)cc3)c3ccc4c(c3)sc3ccccc34)cc2)cc1] 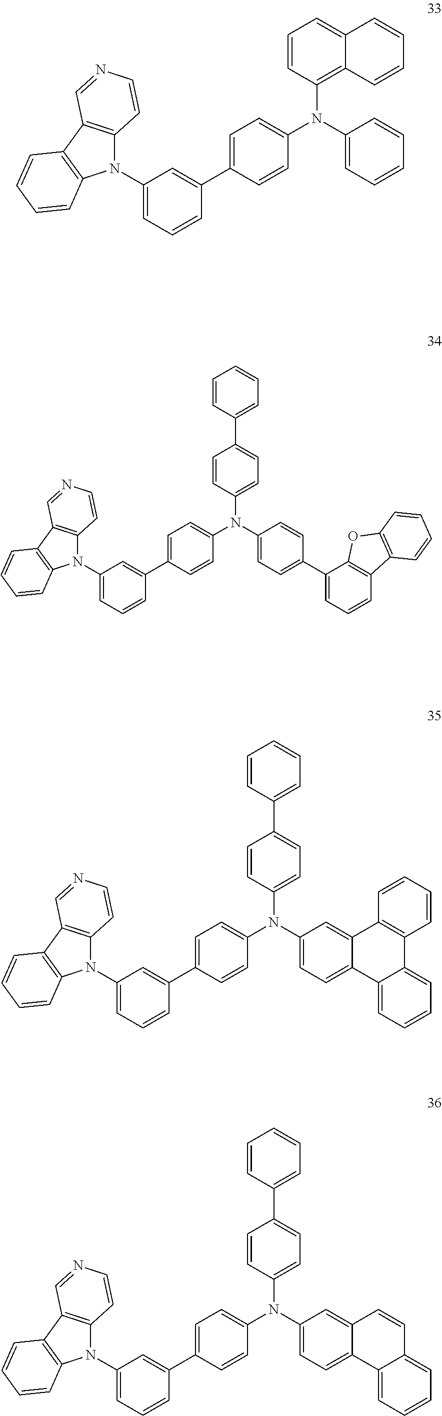 [SMILES: c1ccc(N(c2ccc(-c3cccc(-n4c5ccccc5c5cnccc54)c3)cc2)c2cccc3ccccc23)cc1 ; c1ccc(-c2ccc(N(c3ccc(-c4cccc(-n5c6ccccc6c6cnccc65)c4)cc3)c3ccc(-c4cccc5c4oc4ccccc45)cc3)cc2)cc1 ; c1ccc(-c2ccc(N(c3ccc(-c4cccc(-n5c6ccccc6c6cnccc65)c4)cc3)c3ccc4c5ccccc5c5ccccc5c4c3)cc2)cc1 ; c1ccc(-c2ccc(N(c3ccc(-c4cccc(-n5c6ccccc6c6cnccc65)c4)cc3)c3ccc4c(ccc5ccccc54)c3)cc2)cc1] 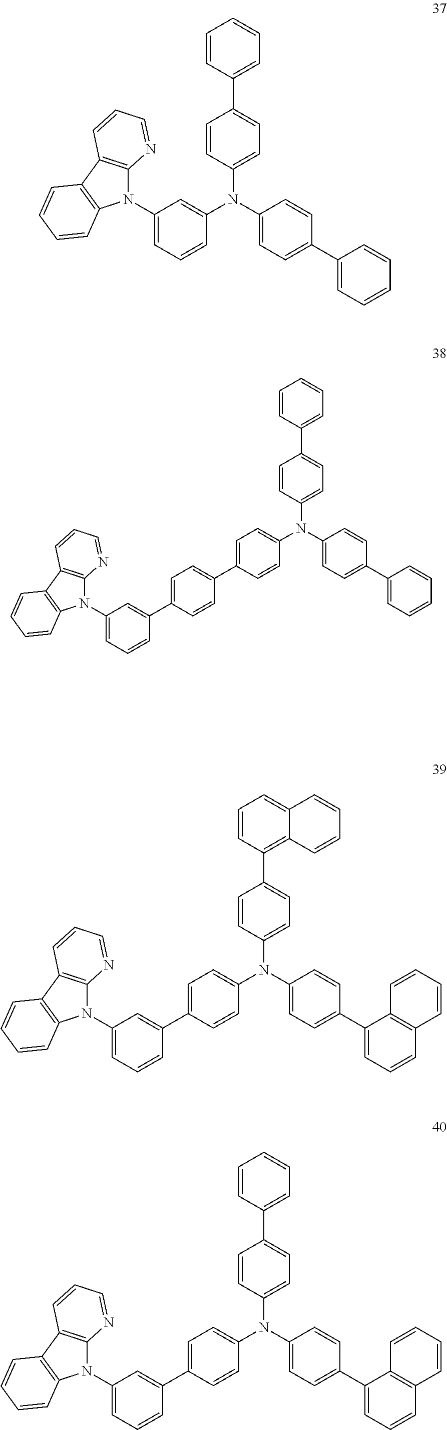 [SMILES: c1ccc(-c2ccc(N(c3ccc(-c4ccccc4)cc3)c3cccc(-n4c5ccccc5c5cccnc54)c3)cc2)cc1 ; c1ccc(-c2ccc(N(c3ccc(-c4ccccc4)cc3)c3ccc(-c4ccc(-c5cccc(-n6c7ccccc7c7cccnc76)c5)cc4)cc3)cc2)cc1 ; c1cc(-c2ccc(N(c3ccc(-c4cccc5ccccc45)cc3)c3ccc(-c4cccc5ccccc45)cc3)cc2)cc(-n2c3ccccc3c3cccnc32)c1 ; c1ccc(-c2ccc(N(c3ccc(-c4cccc(-n5c6ccccc6c6cccnc65)c4)cc3)c3ccc(-c4cccc5ccccc45)cc3)cc2)cc1] 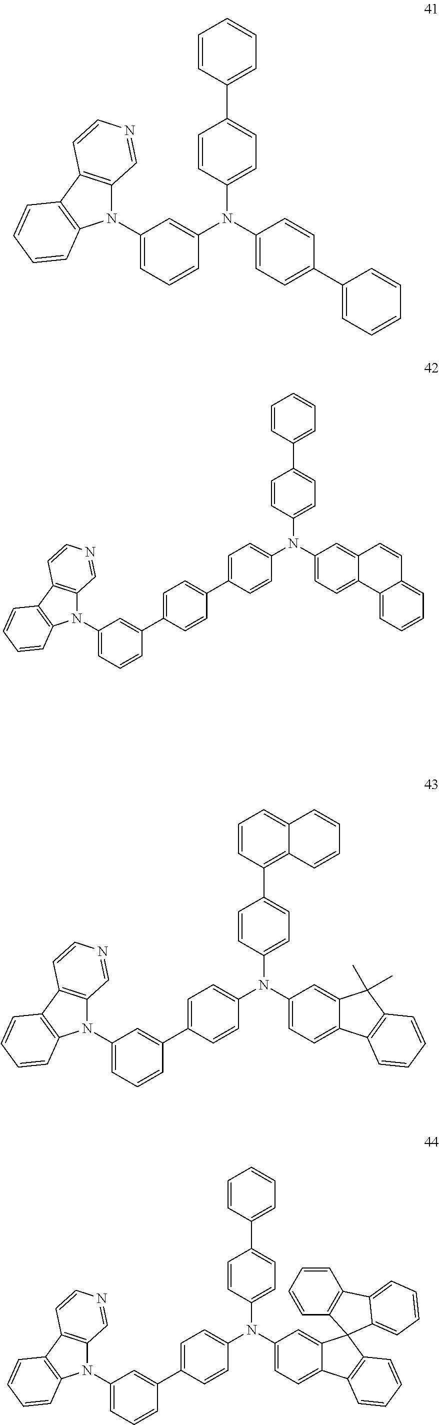 [SMILES: c1ccc(-c2ccc(N(c3ccc(-c4ccccc4)cc3)c3cccc(-n4c5ccccc5c5ccncc54)c3)cc2)cc1 ; c1ccc(-c2ccc(N(c3ccc(-c4ccc(-c5cccc(-n6c7ccccc7c7ccncc76)c5)cc4)cc3)c3ccc4c(ccc5ccccc54)c3)cc2)cc1 ; CC1(C)c2ccccc2-c2ccc(N(c3ccc(-c4cccc(-n5c6ccccc6c6ccncc65)c4)cc3)c3ccc(-c4cccc5ccccc45)cc3)cc21 ; c1ccc(-c2ccc(N(c3ccc(-c4cccc(-n5c6ccccc6c6ccncc65)c4)cc3)c3ccc4c(c3)C3(c5ccccc5-c5ccccc53)c3ccccc3-4)cc2)cc1] 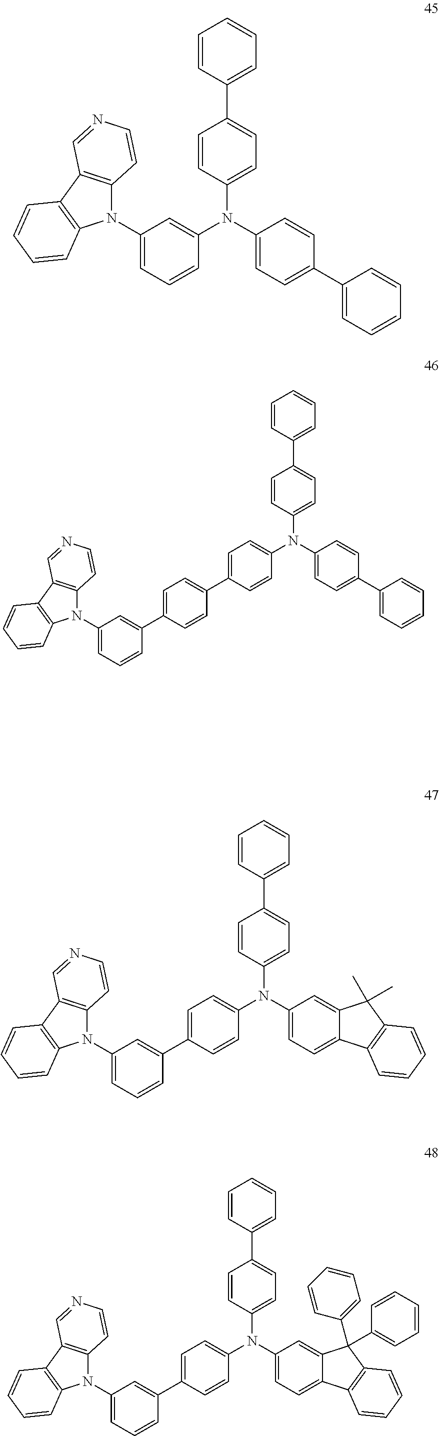 [SMILES: c1ccc(-c2ccc(N(c3ccc(-c4ccccc4)cc3)c3cccc(-n4c5ccccc5c5cnccc54)c3)cc2)cc1 ; c1ccc(-c2ccc(N(c3ccc(-c4ccccc4)cc3)c3ccc(-c4ccc(-c5cccc(-n6c7ccccc7c7cnccc76)c5)cc4)cc3)cc2)cc1 ; CC1(C)c2ccccc2-c2ccc(N(c3ccc(-c4ccccc4)cc3)c3ccc(-c4cccc(-n5c6ccccc6c6cnccc65)c4)cc3)cc21 ; c1ccc(-c2ccc(N(c3ccc(-c4cccc(-n5c6ccccc6c6cnccc65)c4)cc3)c3ccc4c(c3)C(c3ccccc3)(c3ccccc3)c3ccccc3-4)cc2)cc1] 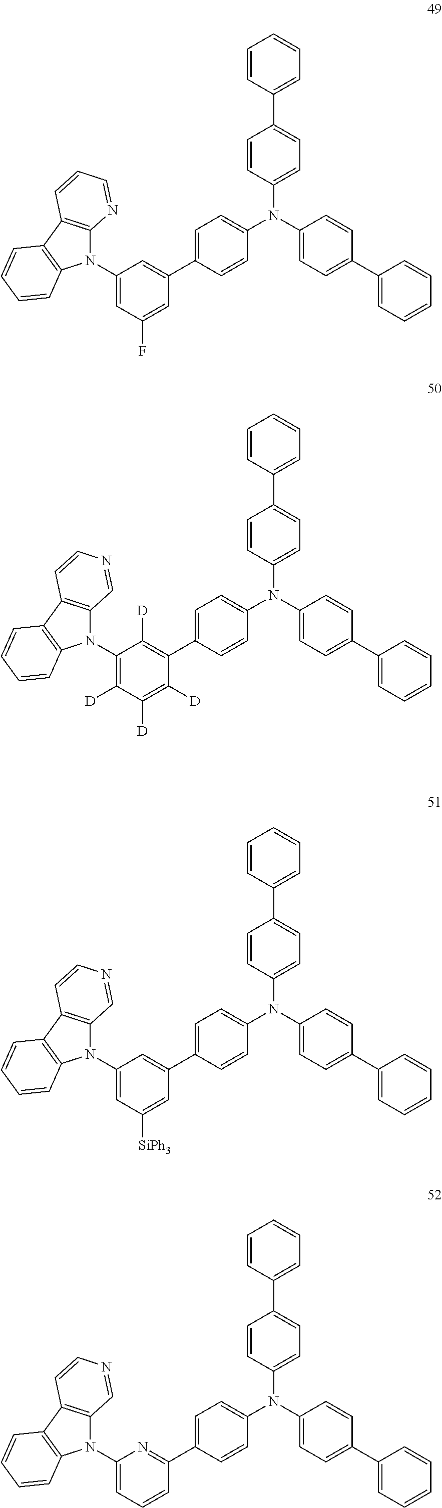 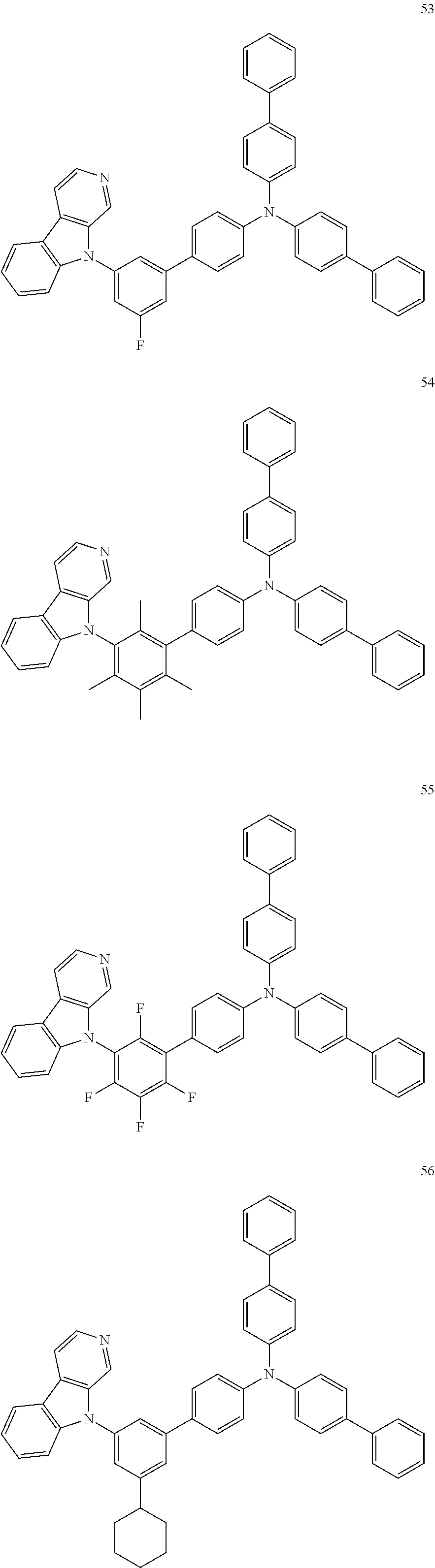 [SMILES: Fc1cc(-c2ccc(N(c3ccc(-c4ccccc4)cc3)c3ccc(-c4ccccc4)cc3)cc2)cc(-n2c3ccccc3c3ccncc32)c1 ; Cc1c(C)c(-c2ccc(N(c3ccc(-c4ccccc4)cc3)c3ccc(-c4ccccc4)cc3)cc2)c(C)c(-n2c3ccccc3c3ccncc32)c1C ; Fc1c(F)c(-c2ccc(N(c3ccc(-c4ccccc4)cc3)c3ccc(-c4ccccc4)cc3)cc2)c(F)c(-n2c3ccccc3c3ccncc32)c1F ; c1ccc(-c2ccc(N(c3ccc(-c4ccccc4)cc3)c3ccc(-c4cc(C5CCCCC5)cc(-n5c6ccccc6c6ccncc65)c4)cc3)cc2)cc1] 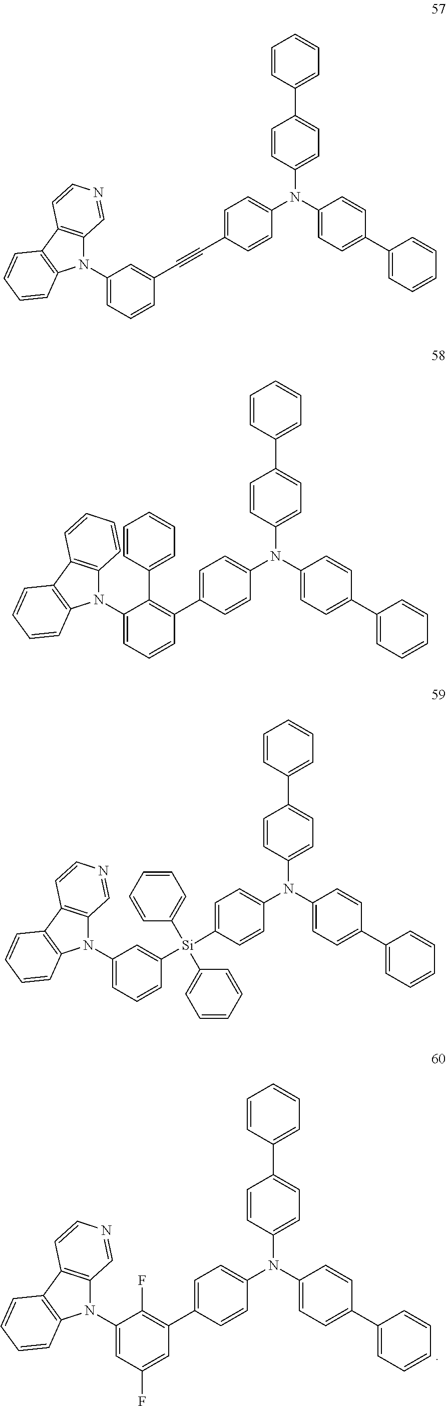 [SMILES: C(#Cc1cccc(-n2c3ccccc3c3ccncc32)c1)c1ccc(N(c2ccc(-c3ccccc3)cc2)c2ccc(-c3ccccc3)cc2)cc1 ; c1ccc(-c2ccc(N(c3ccc(-c4ccccc4)cc3)c3ccc(-c4cccc(-n5c6ccccc6c6ccccc65)c4-c4ccccc4)cc3)cc2)cc1 ; c1ccc(-c2ccc(N(c3ccc(-c4ccccc4)cc3)c3ccc([Si](c4ccccc4)(c4ccccc4)c4cccc(-n5c6ccccc6c6ccncc65)c4)cc3)cc2)cc1 ; Fc1cc(-c2ccc(N(c3ccc(-c4ccccc4)cc3)c3ccc(-c4ccccc4)cc3)cc2)c(F)c(-n2c3ccccc3c3ccncc32)c1]
